(19) United States
(12) Patent Application Publication
(10) Pub. No.: US 2025/0107820 A1
(43) Pub. Date: Apr. 3, 2025

(54) IMAGE-GUIDED ROBOTIC SYSTEM AND METHOD WITH STEP-WISE NEEDLE INSERTION

(71) Applicants: GEORGIA TECH RESEARCH CORPORATION, Atlanta, GA (US); EMORY UNIVERSITY, Atlanta, GA (US); BOARD OF TRUSTEES OF THE UNIVERSITY OF ARKANSAS, Fayeteville, AR (US)

(72) Inventors: Yue CHEN, Atlanta, GA (US); Mishek MUSA, Fayetteville, AR (US); Xiaofeng YANG, Atlanta, GA (US); Nima KOKABI, Atlanta, GA (US); Anthony GUNDERMAN, Atlanta, GA (US)

(21) Appl. No.: 18/729,107

(22) PCT Filed: Jan. 13, 2023
(86) PCT No.: PCT/US2023/010766

§ 371 Date: Jul. 15, 2024

Related U.S. Application Data
(60) Provisional application No. 63/299,304, filed on Jan. 13, 2022.

(51) Int. Cl. A61B 17/34; A61B 34/20; A61B 34/30; A61B 34/32; G06T 7/20

(52) U.S. Cl. CPC A61B 17/3403; A61B 34/32; G06T 7/20; A61B 2017/3407; A61B 2017/3409; A61B 2017/3413; A61B 2034/2063; A61B 2034/2065; A61B 2034/304; G06T 2207/10132; G06T 2207/20084; G06T 2207/30096

(57) ABSTRACT

An exemplary robotic needle insertion system is disclosed that can provide respiration compensated needle insertion for an accurate and efficient ablation, biopsy, draining placement of a needle, among other surgical procedures. The system employs a robotic instrument comprising (i) a needle insertion mechanism configured to deliver the needle insertion in a step-wise manner that mimics the current clinical practice by inserting the needle (e.g., RFA needle) according to respiration and other motion and (ii) a probe manipulation mechanism to direct an ultrasound probe to provide intraoperative image guidance for the needle insertion. The robotic instrument includes a multi-degrees of freedom (DoF) motion control and actuator to provide for the accurate targeting of the needle at any workspace location

### RELATED APPLICATION

This PCT application claims priority to, and the benefit of, U.S. Provisional Patent Application No. 63/299,304, filed Jan. 13, 2022, entitled “Image-Guided Robotic System and Method with Step-Wise Needle Insertion,” which is incorporated by reference herein in its entirety.

### BACKGROUND

Certain medical treatments (ablation, chemotherapy) or diagnosis (biopsies) require the placement of a needle into an organ, tissue, or interstitial spaces within a patient. For example, clinicians would procedurally insert a radiofrequency ablation (RFA) needle into a patient's organ, e.g., for cancer treatment and biopsy. Even with intraprocedural image guidance, the procedure can be technically challenging due to respiration-induced movements of the organ(s). Needle targeting error has been observed to be as large as 5.5 cm in the superior-inferior direction.

Under current procedures or practices, the physician may ask the patient to momentarily hold their breath during the placement of the needle to mitigate such motion. The physician may time (i.e., wait for appropriate motion) the insertion of the needle with the motion of the body while inserting in a stepwise manner to provide for the precise delivery of the needle.

There is a benefit to having improved robotic systems for needle delivery.

### SUMMARY

An exemplary robotic needle insertion system and method are disclosed that are configured to provide respiration-compensated needle insertion of a needle (e.g., biopsy needle or ablation needle), e.g., for an accurate and efficient ablation, biopsy, draining placement of a needle, chemotherapy, among other surgical procedures. The exemplary system employs a robotic instrument comprising (i) a needle insertion mechanism configured to deliver the needle insertion in a continuous or step-wise operation that, with step-wise pauses, mimics the current clinical practice by inserting the needle (e.g., RFA needle) according to respiration and other motion and (ii) a probe manipulation mechanism to direct an ultrasound probe to provide intraoperative image guidance for the needle insertion. To this end, the physician may direct the intended location/end point for the insertion by the controller of the robotic instrument to then execute. The robotic instrument includes a multi-degrees of freedom (DoF) motion assembly and actuator to provide for the accurate targeting of the needle, e.g., via a remote system (e.g., workstation).

The robotic instrument can be sized to be attached to the patient (an animal or a human) for any number of locations, including the abdomen, back, torso, head, pelvic, arm, and legs. The robotic instrument includes straps that can be used to mount the robot onto the patient.

The exemplary robotic needle insertion system may be used for liver ablation, liver biopsy, liver brachytherapy, kidney ablation, and kidney biopsy, lung biopsy, lung ablation, among other procedures.

The robotic instrument, in some embodiments, employs materials compatible with computed tomography (CT) or/and Magnetic Resonance Imaging (MRI) (e.g., metal, plastic) and, in some embodiments, includes or operates with a navigation software system to additionally provide concurrent real-time CT/MRI scans along with the guided ultrasound. In some embodiments, the robotic instrument can be employed in PET or MRI scanner and guided by the PET/MRI scans in the needle insertion. The navigation software can be used to provide real-time, intraoperative, high-resolution image feedback of the needle insertion.

The navigation guidance system, in some embodiments, is configured to first register the real-time ultrasound images to CT images and fuse the ultrasound images to the CT images into a combined output. In some embodiments, individual ultrasound imaging output, CT image output, and fused output may be concurrently presented to the physician through a graphical user interface. The navigation guidance system, in some embodiments, includes a deep learning module configured to extract the dynamic position of tissue of interest (e.g., tumor, cyst, lymph nodes) and provide the dynamic position in the displayed or fused image output in a real-time manner.

As used herein, the term “step-wise” refers to a “move-pause” motion with respect to the insertion of a needle according to a manual insertion protocol in which the clinician would insert a needle when the body and the targeted organ of the body have the least motion and releases the needle when respiration motion is significant.

In some embodiments, the exemplary robotic needle insertion system is configured for optimized, respiration-compensated ablation needle insertion for accurate needle insertion in a dynamically moving target, e.g., within the liver. The exemplary robotic needle insertion system may include a dynamic tumor tracking system to provide tracking of the dynamic tumor position using, e.g., a deep-learning-based feature detection module trained using 3D ultrasound images and/or CT images. The intraoperative 3D ultrasound images are, in some embodiments, registered to the CT images to track the tumors located in the deep region of the body.

In an aspect, a system is disclosed comprising a robotic instrument comprising a needle (e.g., ablation needle, biopsy needle) to be employed in a procedure; a multi-degree of freedom motion control and actuator assembly comprising a base and a movable platform coupled to the base via a set of two or more actutate-able linkages; and a needle insertion mechanism coupled to a base of the multi-degree of freedom motion control and actuator assembly, the needle insertion mechanism being configured to (i) rotate two or more cams or rollers to move the needle, during a sensor-detected period of rest, along a delivery axis in a controlled step-wise manner that advance the needle into a pre-defined target and (ii) halt rotation of the two or more cams or rollers during a period of sensor-detected body movement.

In some embodiments, the robotic instrument comprises a controller, wherein the controller is configured to direct (i) the rotation of the two or more cams or rollers to move the needle, during a sensor-detected period of rest, along the delivery axis in a controlled step-wise manner that advance the needle toward the pre-defined target and (ii) halting of the rotation of the two or more cams or rollers during a period of sensor-detected body movement.

In some embodiments, the robotic instrument includes a central body that mounts a probe manipulation mechanism to direct an ultrasound probe to provide intraoperative image guidance for the needle insertion.

In some embodiments, the needle insertion mechanism is mounted to the central body.

In some embodiments, at least one of the two or more cams or rollers is adjustable to accept different needles of different diameters.

In some embodiments, the robotic instrument is made of a material that is compatible with an X-ray scanner, MRI, or CT scanner.

In some embodiments, the robotic instrument is sized to be attached to a subject (e.g., animal or human), including at least one of an abdomen region, a back region, a torso region, a head region, a pelvic region, an arm region, and a leg region.

In some embodiments, the needle insertion mechanism comprises a motor (e.g., stepper, DC, AC motor) configured with PID controls.

In some embodiments, the system further includes a navigation system configured to connect to a robot control module over a high-speed communication channel via a data communication protocol, wherein the robot control module is configured with drivers to actuate one or more motors of the needle insertion mechanism.

In some embodiments, the navigation system includes a deep-learning-based model (e.g., regional convolutional neural network (RCNN)) that integrates an attention-aware long short-term memory (LSTM) framework trained with low contrast and SNR ultrasound images.

In another aspect, a method is disclosed comprising positioning, by a processor, a needle insertion mechanism via multi-degree of freedom motion control to orient a needle for insertion into a subject; tracking, by the processor, tissue or tumor in an organ of the subject using a tracking operation based on acquired ultrasound images acquired at an insertion area of the needle; and delivering, by the processor, the needle into the subject in a stepwise manner by rotating two or more cams or rollers of the needle insertion mechanism to move the needle along a delivery axis in stepwise increments, wherein each incremental delivery is based on the tracking.

In some embodiments, the method further includes directing (i) the rotation of the two or more cams or rollers to move the needle, during a sensor-detected period of rest, along the delivery axis in a controlled step-wise manner that advances the needle toward the pre-defined target and (ii) halting of the rotation of the two or more cams or rollers during a period of sensor-detected body movement.

In some embodiments, the robotic instrument includes a central body that mounts a probe manipulation mechanism to direct an ultrasound probe to provide intraoperative image guidance for the needle insertion.

In some embodiments, the needle insertion mechanism is mounted to the central body.

In some embodiments, at least one of the two or more cams or rollers is adjustable to accept different needles of different diameters.

In some embodiments, the robotic instrument is made of a material that is compatible with an X-ray scanner, MRI, or CT scanner.

In some embodiments, the robotic instrument is sized to be attached to a subject (e.g., animal or human), including at least one of an abdomen region, a back region, a torso region, a head region, a pelvic region, an arm region, and a leg region.

In some embodiments, the needle insertion mechanism comprises a stepper motor configured with PID controls.

In some embodiments, the method further includes receiving target coordinates from a user interface for the insertion of the needle into the subject; executing a control loop that (i) senses a period of rest or motion of the subject, (ii) directs the rotation of the two or more cams or rollers to move the needle, during the sensor-detected period of rest, in the controlled step-wise manner that advance the needle toward the target coordinates and (ii) directs halting of the rotation of the two or more cams or rollers during a period of sensor-detected body movement; tracking an end point or a landmark of the needle, or an associated assembly; and exiting the control loop upon the end point or the landmark reaching the target coordinates.

In another aspect, a non-transitory computer-readable medium is disclosed having instructions stored thereon, wherein execution of the instructions by a processor causes the processor to control the system or method of any one of the above-discussed claims.

### BRIEF DESCRIPTION OF THE DRAWINGS

FIG. 1 shows an example of the robotic needle insertion system 100 (shown as 100a) configured to respiration-compensated needle insertion in accordance with an illustrative embodiment.

FIGS. 2A-2E (“FIG. 2”) and FIGS. 3A-3C (“FIG. 3”) each shows an embodiment of the robotic instrument 102 (shown as 102a and 102b, respectively) in accordance with an illustrative embodiment.

FIG. 4 shows an example clinical workflow 400 for the robot instrument (e.g., 200, 300) in accordance with an illustrative embodiment.

FIG. 5A shows views of the robotic instrument, e.g., for abdominal radiofrequency ablations, biopsies, or other treatments or procedures.

FIG. 5B shows an illustrative example of a real-time tracking operation.

FIG. 6A shows an experimental setup for the dynamic ex-vivo study.

FIG. 6B shows a comparison of positional error (left) for the uncompensated motion (UMPE) and compensated motion (CMPE) case and orientation error (right) for the uncompensated motion (UMOE) and compensated motion (CMOE) case of the dynamic ex-vivo targeting experiment.

FIGS. 6C and 6D show the experimental setup for the animal-based experiment employing a prototype of the exemplary robotic instrument.

FIG. 6E shows experimental results comparing the outputs of a force model to experimental results.

FIG. 6F shows pressure data recorded using a pressure sensing array and corresponding displacement position commands sent to the stepper motors for the dynamic ex-vivo liver test.

FIG. 6G shows the results of a free space accuracy validation.

### DETAILED SPECIFICATION

To facilitate an understanding of the principles and features of various embodiments of the present invention, they are explained hereinafter with reference to their implementation in illustrative embodiments.

### Robotic Needle Insertion System

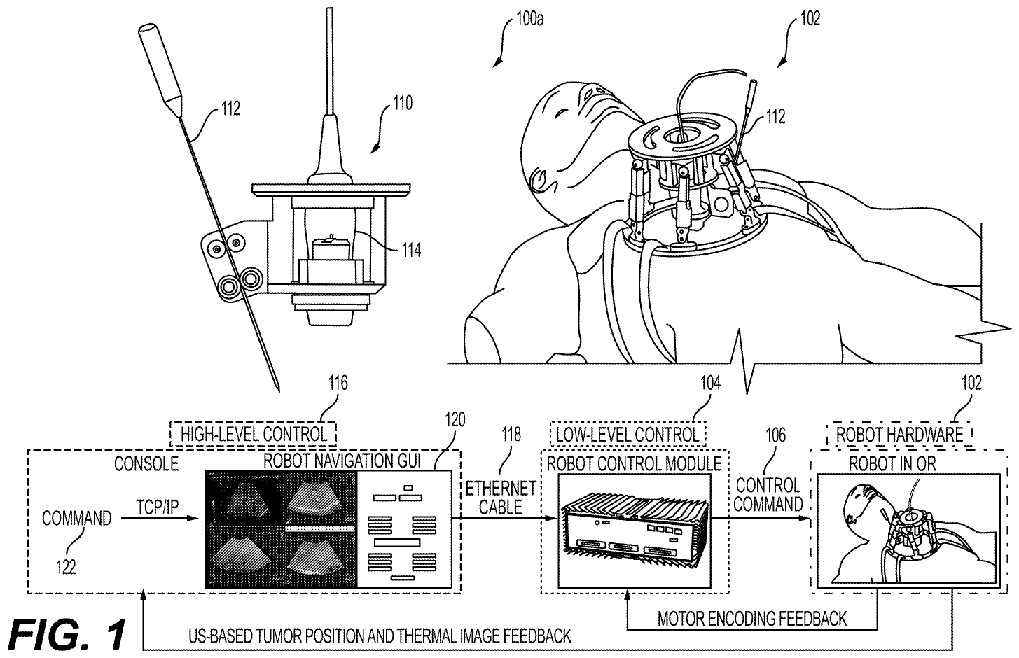

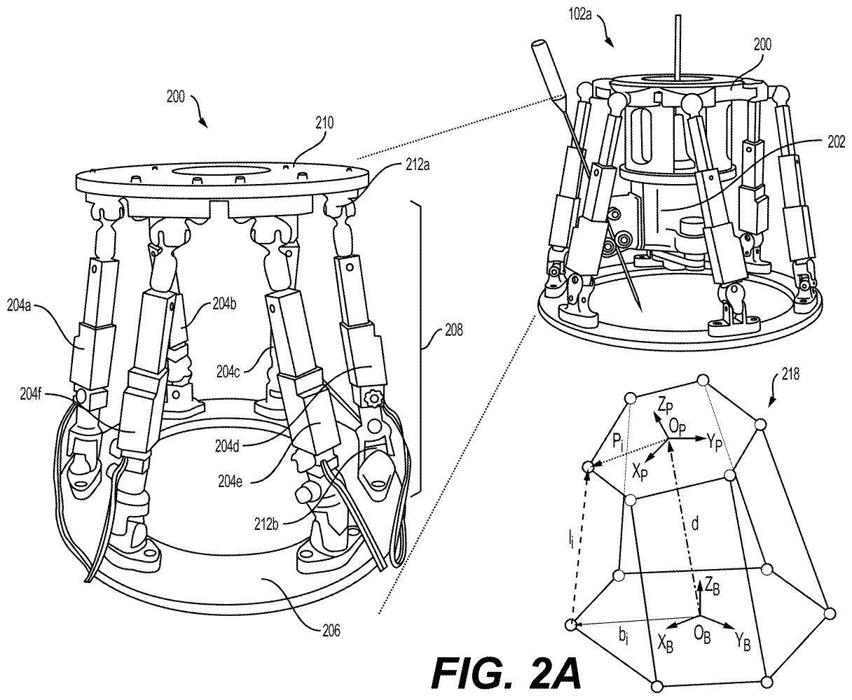

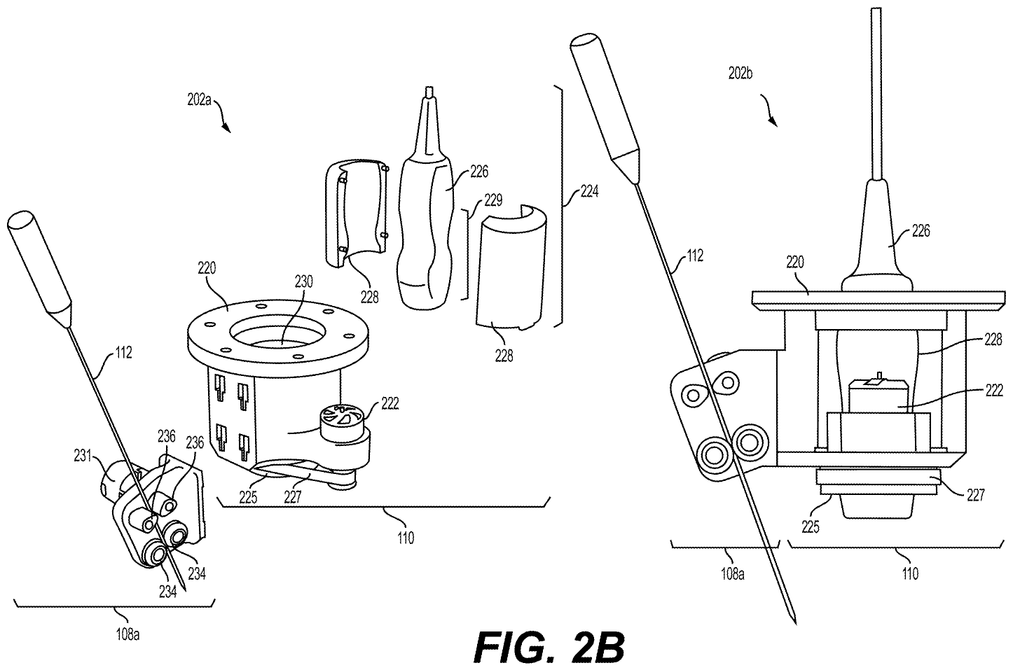

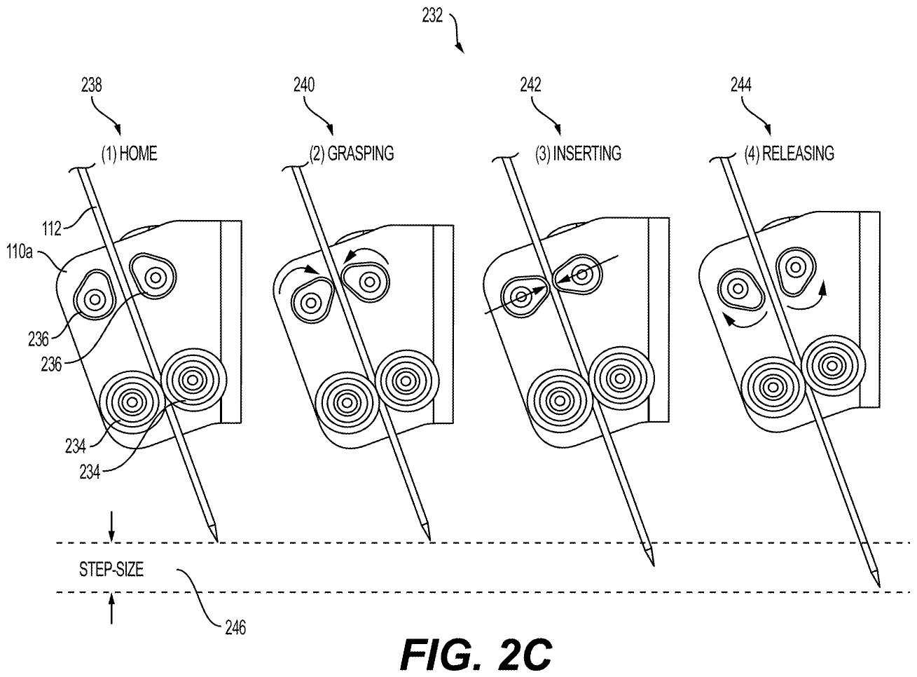

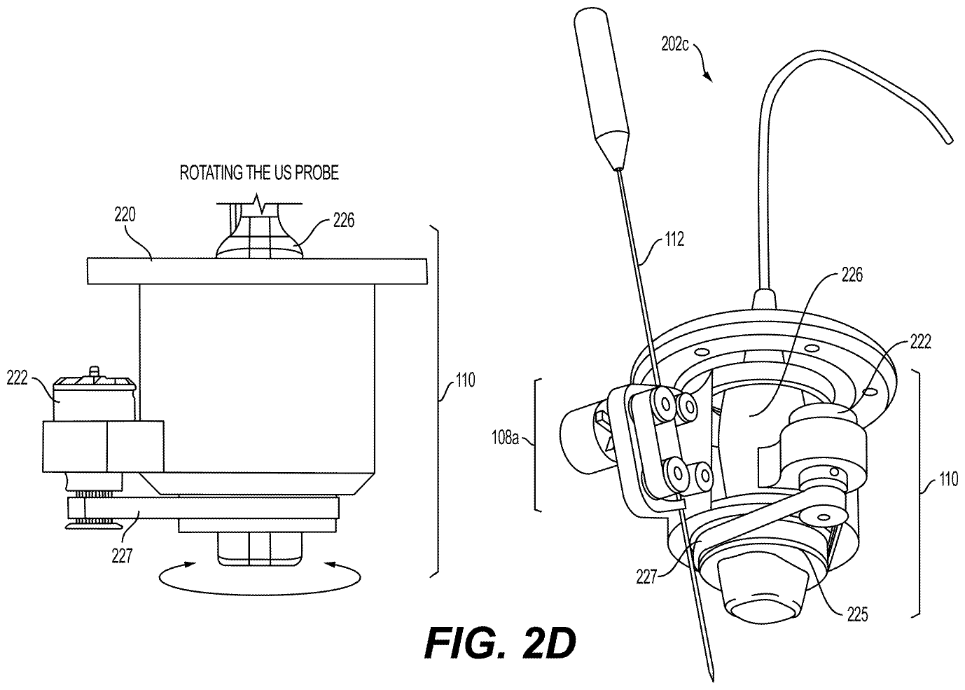

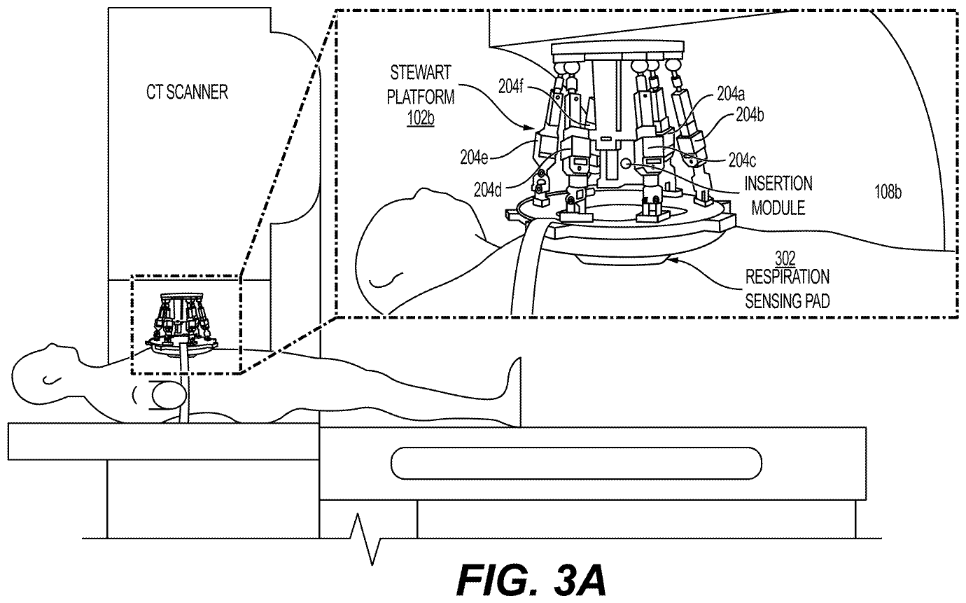

FIG. 1 shows an example of the robotic needle insertion system 100 (shown as 100a) configured to respiration-compensated needle insertion in accordance with an illustrative embodiment. The robotic needle insertion system 100a includes a robotic instrument 102 (shown as “Robot hardware” 102) that connects to a robot control module 104. The robot control module 104 provides control command 106 to a motorized needle insertion assembly 108 (not shown-see FIGS. 2B, 3C) and a motorized probe manipulation assembly 110 (not shown-see FIGS. 2 and 3) to perform the needle insertion in a continuous or step-wise manner that with step-wise pauses mimic the current clinical practice by inserting the needle 112 (e.g., RFA needle) according to respiration and other motion while guided by a mounted ultrasound transducer 114 that provides intraoperative image guidance for the needle insertion.

In the example shown in FIG. 1, the robotic needle insertion system 100a includes a robot navigation system 116 that operatively connects to the robot control module 104 over a high-speed communication channel 118. The robot navigation system 116 is configured to execute a robot navigation GUI 120 to provide scan images of the ultrasound probes 114 and to receive commands 122 for the robotic instrument 102. The ultrasound probe and robot navigation system can provide an update rate of 50 Hz.

The robotic instrument 102 is configured to provide manipulation control in pre-defined increment, e.g., in 0.5° increment, or other described herein in orienting the needle 112 and in pre-defined stepwise-pause delivery (e.g., 1 mm stepwise) in translation.

### Example Robotic Instrument

FIGS. 2A-2E (“FIG. 2”) and FIGS. 3A-3C (“FIG. 3”) each shows an embodiment of the robotic instrument 102 (shown as 102a and 102b, respectively) in accordance with an illustrative embodiment. In FIG. 2, the robotic instrument 102a includes a motorized probe manipulation assembly 110 and a step-wise needle insertion assembly 108a, collectively configured to deliver the needle insertion in a step-wise manner via its mechanical actuation to provide step-wise pauses in the insertion of the needle 112. In FIG. 3, e.g., see FIGS. 3A, the robotic instrument 102 (shown as “Stewart Platform” 102b) includes a motorized continuous needle insertion assembly 110b (shown as “Insertion Module” 110b) configured to via precise controls to provide the needle insertion with step-wise pauses. The motorized continuous needle insertion assembly 108b of FIG. 3 can be similarly configured to operate with the motorized probe manipulation assembly 110 of FIG. 2.

In FIGS. 2 and 3, the robotic instruments 102a, 102b each include a multi-degrees of freedom (DoF) motion control and actuator system 200, 300 that is coupled to the integrated motorized needle insertion assembly 108 (shown as 108a, 108b, respectively) and the motorized probe manipulation assembly 110 (see FIG. 2B).

Multi-DOF motion control and actuator system. Each of FIGS. 2A and 3A shows an example of the multi-degrees of freedom (DoF) motion control and actuator system 200, 300. The example of FIGS. 2A and 3A, the multi-DOF motion control and actuator systems 200, 300 each include 6 actuators 204 (shown as 204a, 204b, 204c, 204d, 204e) that can provide 6 DOF control. Other configurations and number of actuators (e.g., 3, 4, 5, 6, 7, 8) may be employed. The multi-DOF control and actuator system 200, 300 includes a base platform 206 that connects, via actuatable linkages 208, to an assembly platform 210. In FIG. 2, the assembly platform 210 is fixably connected to the assembly 202 comprising the integrated motorized needle insertion assembly 108a and the motorized probe manipulation assembly 110 and is manipulate-able via actuation of the actuatable linkages 208 that extend and contract by the actuator 204 to direct orient the assembly 202 for the needle insertion. In FIG. 3, the assembly platform 210 is fixably connected to a motorized needle insertion assembly 108b and is manipulate-able via actuation of the actuatable linkages 208 that extend and contract by the actuator 204 to direct orient the motorized needle insertion assembly 108b for the needle insertion.

As shown in FIG. 2A, the actuatable linkages 208 are operatively connected at its respective ends to a hinge assembly 212 (shown as 212a and 212b, FIG. 2A) that connects to the top-side of the base platform 206 and the under-side of the assembly platform 210. Diagram 218 (later discussed) shows a linkage model of the multi-DOF motion control and actuator system 200, 300. In FIG. 2, to minimize the robot dimension, the multi-DOF control and actuator system 200 includes six Squiggle linear piezoelectric motors (manufactured by New Scale Technologies, Victor, NY). Other linear actuators and motors may be employed.

FIG. 2B are diagrams showing the assembly 202 (shown as 202a for the unassembled view and 202b for the assembled view) that includes the integrated motorized needle insertion assembly 108a and a motorized probe manipulation assembly 110. The assembly 202 is mounted onto the manipulated platform 210 of the actuators of the multi-DoF motion control and actuator system 200.

As shown in the example of FIG. 2B, the motorized probe manipulation assembly 110 of assembly 202 includes a central body 220 that houses a motor 222 for an ultrasound probe manipulation assembly 224. The central body 200 is cylindrically shaped, in this example, and includes a central hollow space for the placement of an ultrasound probe manipulation assembly 224. The ultrasound probe manipulation assembly 224 includes an ultrasound probe 226 that is maintained in the motorized probe manipulation assembly 110 by a probe housing 228 that couples over the handle region 229 to retain the ultrasound probe 226 in the manipulate-able recess 230 centrally formed in central body 220. The probe housing 228 can be 3D printed to fit the contoured shape of the transducer probe 226 to retain it. The probe housing 228 may be generally flat on the outside surface to provide an even surface for the manipulation of the ultrasound transducer probe by the motor 222. The motorized probe manipulation assembly 110 includes a rotatable probe housing body 225 that couples to the motor 222 over a belt connection 227. FIG. 2D shows the operation of the motorized probe manipulation assembly 110 of the assembly 202 (shown as 202c). In the example shown in FIG. 2D, the motor 222 is a DC motor (EC-max 16, manufactured by Maxon) that is connected via a shaft to a belt configured to rotate the probe housing 228. The motorized probe manipulation assembly 110 provides a 7^{th} degree of freedom control for the system. In other embodiments, the system can be configured with additional motors to provide an additional degree of freedom of manipulation of the transducer probe 226. A conventional or custom ultrasound transducer probe can be used.

The central body 220 is fixably coupled to the motorized needle insertion assembly 108a. The motorized needle insertion assembly 108a includes a base structure that houses a motor 231 configured to actuate the needle driving mechanisms (e.g., 236) comprising two guiding rollers 234 and two cam rollers 236.

In FIG. 3A, the robot instrument 300 includes the multi-DOF motion control and actuator system (e.g., stewart platform [23], [24]), a motorized needle insertion assembly 108 as a roller insertion module, and a respiration sensing pad 302. The Stewart platform includes six linear actuators (e.g., L12-30-50-6-R, manufactured by Actuonix Motion Devices, Canada) connected to a lower platform via a universal joint (e.g., 103.09.2020, manufactured Huco, England) and an upper platform via an upper ball-joint.

In the example shown in FIG. 3A, the motorized needle insertion assembly 108b as an insertion module includes a one-degree-of-freedom friction drive roller mechanism that can provide for insertion and retraction of the needle 112 (e.g., ablation needle, etc.). The motorized needle insertion assembly 108b is mounted to the upper platform so that the needle 112 can be concentric with the upper platform.

The respiration sensing pad 302 may be a molded pressure sensing apparatus (e.g., made of silicone) that is attached to the base of the lower platform. FIG. 3B shows an example manufacturing operation of the respiration sensing pad 302. In the example shown in FIG. 3B, the operation employs a three-part molding process with silicone mold-making rubber (Rebound 25, Smooth-On, USA), ensuring patient comfort when the robot is placed on the patient. In FIG. 3B, pane A, the silicone is poured into the first mold. In pane B, the second mold is then located concentrically on the first mold, creating the pressure-sensing cavity. In pane C, the bottom molded part is removed from the molds. In pane D, the third mold is then assembled with pneumatic tubing located inside, and the silicone is poured. In pane E, the silicone cures around the tubes. (F) A thin layer of silicone is then poured, and the first cast is located on top of the second cast. In pane G, the finished molded part is then removed from the mold. Pane H shows the inner channels after the molding process. The respiration sensing pad 302 may include cells that are connected to a pressure sensor using pneumatic tubing.

Due to the patient mounted design, when respiration occurs, the abdomen expands and respiration-induced deformation in the pressure cells occurs. This deformation results in an increase in the pressure within the cell, allowing the implementation of the RMCP. The lower platform has slots for adjustable belts that are used for strapping the robot to the patient. This allows the clinician to position the robot in any manner to provide a desired entry point for the RFA procedure. The body-mounted design allows the robot to move up and down with respiration, while the Stewart platform enables the entry location and vector to change based on the patient's respiration. The base of the robot platform has asymmetric points designed into the base for rigid point registration [26].

Example Motorized Needle Insertion Assembly Operation #1. FIG. 2C shows a partial view of the motorized needle insertion assembly 108a and its operation to provide an example of the step-wise insertion operation 232. In the example shown in FIG. 2C, the motorized needle insertion assembly 108a includes the (2) guiding rollers 234 and the (2) cam rollers 236 configured to grasp and retain the needle 112 to provide a cam mechanism that can move the needle 112 in a stepwise motion. The non-grasp configuration is shown as position “1—Home” 238. Position 2 “Grasping” (240) shows the needle 112 in an insertable position in which the cam rollers 236 are engaged to retain the needle 112 in a given orientation. The positioning of the needle 112 can be manipulated by changing the positioning of the cam roller accordingly, while the orientation of the needle 112 can be manipulated by the multi-DOF motion control and actuator system 200. At least one of each of the cam rollers 236 and guiding rollers 234 may be constructed of a conformable material (e.g., rubber) to provide grasping friction for the needle 112.

When the needle 112 is oriented to its desired position, e.g., via the multi-DOF motion control and actuator system 200, the cam roller 236 can be actuated (see position “3-Inserting” (242) and position “4-releasing” (244)) in a stepwise manner to deliver the needle into the subject for a pre-defined distance 246. The 2 guiding rollers (234) and 2 cam rollers (236) can be adjusted to vary the driving clearance of the needle 112 to accommodate needles having a thickness from 0 mm diameter to 5 mm. FIG. 2C shows a 1.7 mm diameter needle (for scaling).

Example Motorized Needle Insertion Assembly Operation #2. FIG. 3C shows another embodiment of the motorized needle insertion assembly 108 (shown as 108b) as a needle delivery system in which the step-wise motion and motion compensation is entirely electronically controlled. In the example shown in FIG. 3C, the motorized needle insertion assembly 108b includes two driving rollers 310 (shown as 310a, 310b) and two guiding rollers 312 (shown as 312a, 312b) that each remains in continuous contact with the needle 12.

The needle 112 is inserted through the guiding rollers 312 to ensure that the needle is centered with respect to the driving and guiding rollers. An example of the guiding roller 312 is a square profile o-ring (e.g., model 1171N199, manufactured by McMaster, USA) that is adhered to an outer race of a deep-groove ball bearing (e.g., model MR105-2RS, manufactured by Uxcell, China) using cyanoacrylate. Other configurations and guides may be used.

The driving rollers 310 may use the square profile o-ring as attached to meshed spur gears with a custom-designed hub that incorporates the o-ring. The meshed spur gears can provide the driving force to the needle through friction. One or more driving gear may be directly linked to a stepper motor through a keyed shaft.

The system includes a motor driver (not shown) that is configured to enable and disable power to a 222 (shown as 222a). In the example of FIG. 3C, the driving motor 222a is a NEMA 17 stepper motor. Other types of actuators or motors may be used, e.g., linear actuators, DC motors, and AC motors, among others described herein. When power is enabled, the needle is grasped (i.e., maintained its position via the motor's fixed position); when the power is disabled, the needle is released (i.e., allowed to move as the motor is allowed to move). For insertion, the power is enabled, and the needle is driven using the motor 222a. For motion compensation, power is disabled (and the needle is allowed to move in conjunction with the tissue and body-motion-induced movements).

The hardware components may be constructed based on a modular approach to allow a fast setup in the operating environment, thereby reducing the interventional procedure time. The modular design concept facilitates the re-use of the parallel robot and employs an automatic needle insertion unit that is disposable. The exemplary robot system can be secured to the patient's abdomen with adjustable straps in practical applications.

Force Modeling of Roller Insertion. FIG. 3C shows modeling of the force to be applied to the needle body caused by the deformation of the rubber roller induced by the interference between the needle body and the roller body. The force modeling can provide the needle insertion force of the roller operation.

Drawings from other minimally invasive applications [33], [34], Castigliano's first theorem may be used to evaluate the normal force applied to the roller based on the roller's deformation per Equation 1.

$$F = \frac{\partial U}{\partial δ} \qquad ( Eq . 1 )$$

In Equation 1, F is the force applied by the roller, U is the strain energy in the roller caused by the deformation, and δ is the gap between the rollers, which defines the roller deformation based on geometry. Because of the symmetry, the deformed volume of the roller can be defined in four different segments (see volume 314). To solve for the strain energy in this volume as a function of δ, it can be first recognized that the strain in a roller fiber perpendicular to the needle body, as represented by the black hashed line in the volume 314, can be defined per Equation 2.

$$ε = \frac{{y}_{0} - y}{{y}_{0}} \qquad ( Eq . 2 )$$

In Equation 2, & is the strain in the fiber, y₀ is the initial fiber length, and y is the fiber length after deformation. The initial fiber length varies as a function of x defined per Equation 3.

$${y}_{0} ( x ) = \sqrt{{r}_{r , o}^{2} - {x}^{2}} - \sqrt{{r}_{r , i}^{2} - {x}^{2}} \qquad ( Eq . 3 )$$

In Equation 3, r_{r,o} is the outer radius of the roller, r_{r,i} is the inner radius of the roller, and x is the displacement from the start of the volume to the end of the volume in the transverse view of the roller/needle pair. The start of the volume in the transverse view occurs at the line intersecting the center of both rollers. The end of the volume in the transverse view can be defined by the gap per Equation 4.

$$l ( δ ) = \frac{{δ}^{2}}{4} - {r}_{t , o}^{2} + 2 {r}_{t , o} {r}_{r , o} + δ {r}_{t , o} - δ {r}_{r , o} \qquad ( Eq . 4 )$$

In Equation 4, r_{t,o} is the outer radius of the tube, and l is the end of the volume in the transverse view of the roller/needle pair. The fiber length after deformation can be defined by Equation 5.

$$y ( x , t ) = {y}_{o} ( x ) + \frac{δ}{2} - \sqrt{{r}_{t , o}^{2}} - {t}^{2} \qquad ( Eq . 5 )$$

In Equation 5, t is the displacement from the start of the volume to the end of the volume in view of the longitudinal cross-section of the roller/needle pair. The start of the volume in the longitudinal view occurs at the line intersecting the center of both rollers. The end of the volume in the longitudinal view is defined by the gap per Equations 6 and 7.

$${t}_{max} ( δ , x ) = \sqrt{{r}_{t , o}^{2} - {a ( δ , x )}^{2}} \qquad ( Eq . 6 )$$

$$a ( δ , x ) = {r}_{r , o} + \frac{δ}{2} - \sqrt{{r}_{r , o}^{2} - {x}^{2}} \qquad ( Eq . 7 )$$

In Equation 6, tₘₐₓ is the end of a slice of the volume in the longitudinal view of the roller/needle pair. The strain energy in the entire deformed volume of one roller given some gap δ can be written per Equations 8 and 9.

$$U = 4 {\int }_{0}^{l ( δ )} {\int }_{0}^{{t}_{max} ( δ , x )} {\int }_{0}^{y ( x , t )} W ( ε ) , dydtdx \qquad ( Eq . 8 )$$

$$W ( ε ) = {\int }_{0}^{ε} σ ( e ) de \qquad ( Eq . 9 )$$

In Equations 8 and 9, W is the strain energy density in the fiber. The stress is dependent on the modulus of elasticity of the roller, which can be approximated using the relationships in [36]. Thus, the friction force inserting the needle, applied by both rollers, can be determined per Equation 10.

$${F}_{frictionn} = 2 μ \frac{\partial U}{\partial δ} \qquad ( Eq . 10 )$$

In Equation 10, μ is the coefficient of static friction, which can be set as 0.55 for the primary tube used.

### Example Respiration Motion Compensation Protocol (RMCP)

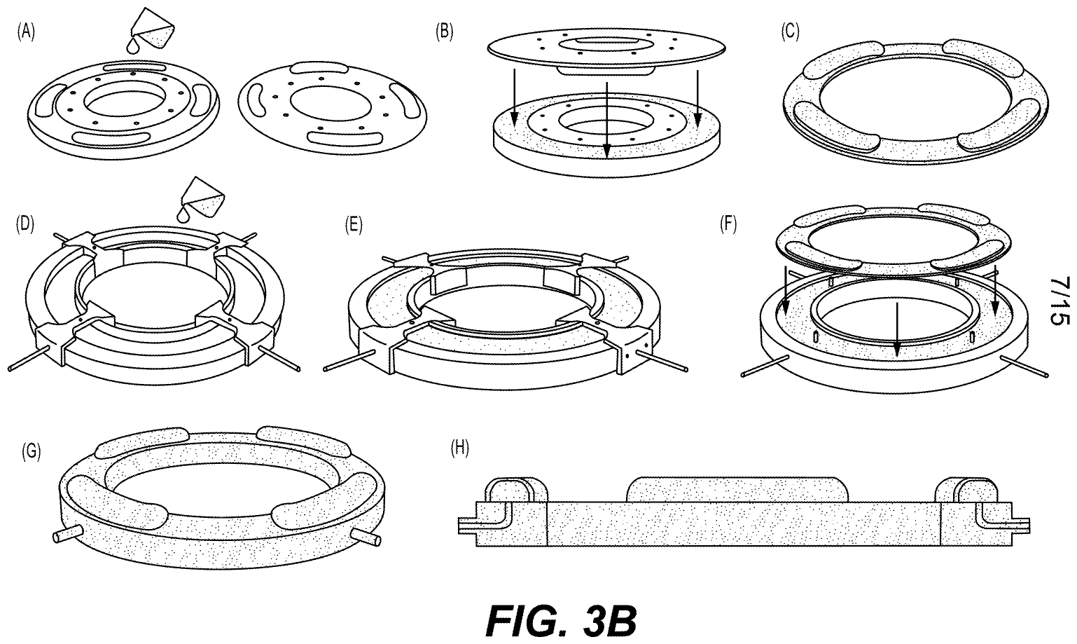

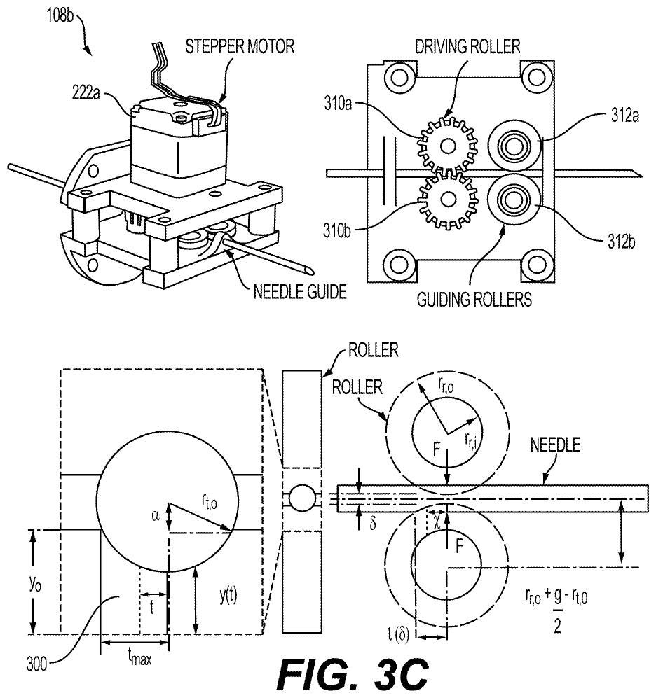

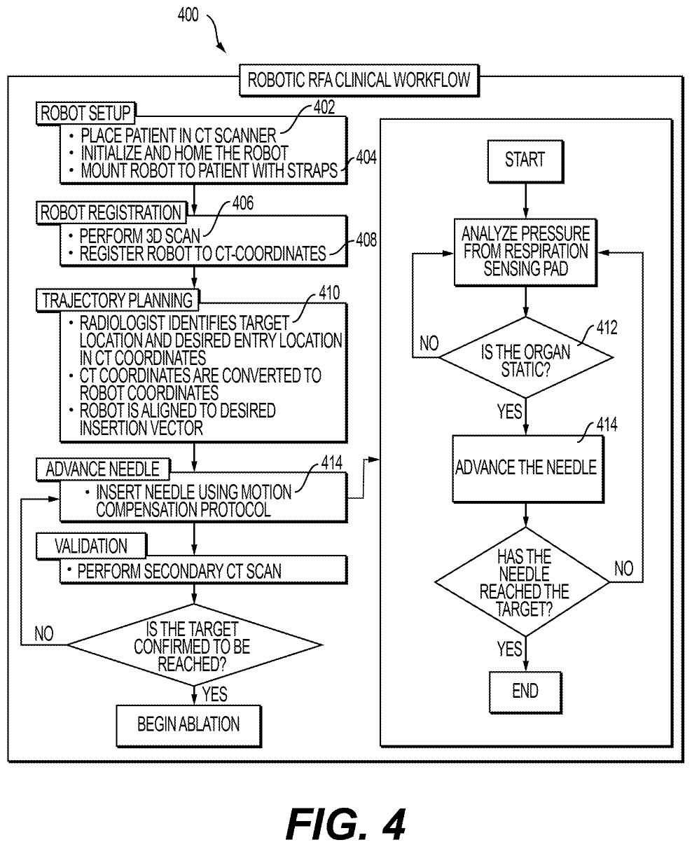

FIG. 4 shows an example of a clinical workflow 400 for the robot instrument (e.g., 200, 300). In the example shown in FIG. 4, the patient may be first positioned (402) in a CT scanner, and the robot instrument (e.g., 200, 300) may be mounted (404) to the patient (e.g., patient's abdomen) using adjustable straps. An initial CT scan may be performed (406) during the static portion of the respiration cycle to register the robot to the CT scanner using the point-based registration method (408). This same scan may be used to obtain the desired target location. The radiologist may define (410) a desired entry location, and the robot may align the needle insertion module to the desired insertion vector.

During ablation needle advancement, the respiration sensing pad may continuously track (412) the patient's respiratory cycle. This may allow motion compensation detection while avoiding the need to continuously apply radiation to the patient for needle tracking. Drawing from current clinical practice, the robot may advance (414) the needle during the static portion of the respiration cycle. When breathing is detected, the robot may “release” the needle by disabling motor power, allowing the needle to move freely with the liver.

### Example Operation

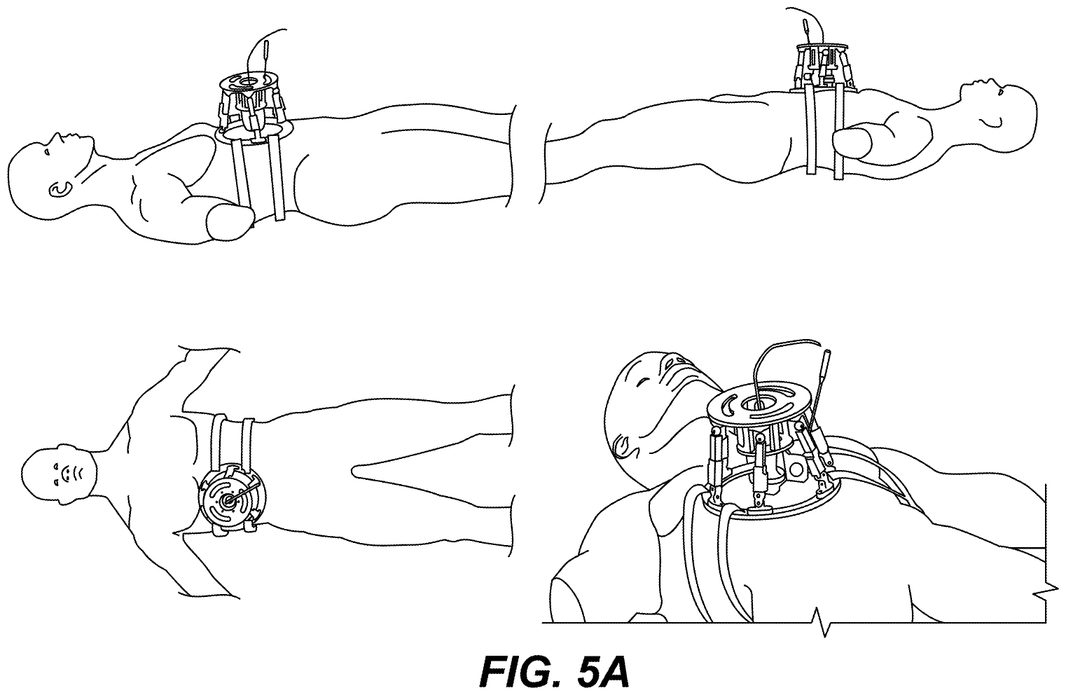

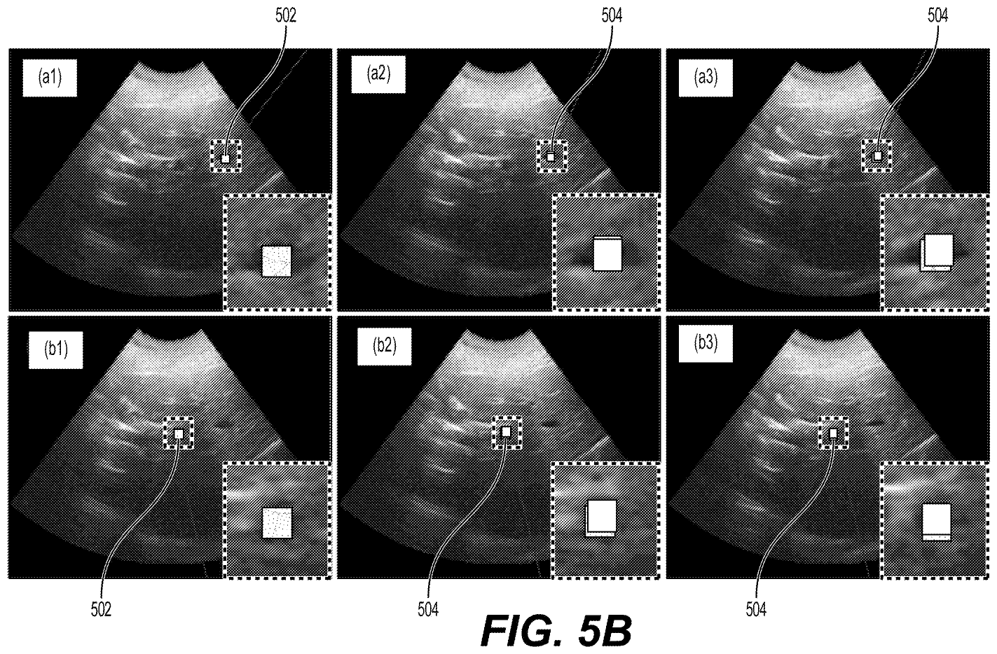

FIG. 5A shows views of the robotic instrument, e.g., for abdominal radiofrequency ablations, biopsies, or other treatments or procedures. Radiofrequency ablation (RFA) a thermal therapy that is used to induce coagulative necrosis, is an effective minimally-invasive treatment used for a variety of solid tumor cancers, including lung, breast, kidney, pancreatic, and liver.

### Example Navigation Guidance

The robot navigation system may include a robot control algorithm and control electronics to provide visualization of the orientation/insertion of the needle towards a target within the moving organ (e.g., an ablation needle into a tumor in the liver). The robot navigation system may employ a robot kinematic model and path planning algorithm, e.g., based on the method described in [19′]. The robot navigation system may include a D.C. motor and piezoelectric motor control, e.g., comprising a PID (proportional-integration differentiation) controller to execute a calculation rate of about 1 KHz.

The robot navigation system is configured to connect to the robot control module over a high-speed communication channel via a data communication protocol.

The robot navigation system may be configured with a deep-learning-based model (e.g., regional convolutional neural network (RCNN)) that integrates an attention-aware long short-term memory (LSTM) framework trained with low contrast and SNR ultrasound images. Patient-specific anatomical features can be extracted from input 2/3D ultrasound images to train the LSTM using the attention-based feature selection process. For a given sequence of ultrasound frames {I₁, I₂, . . . , Iₙ}, an object (tumor) L₁ of the first frame is manually contoured via MRI/CT-US registration procedure [22′]-[26′] to track the tumor and predict its location {L*₂, L*₃, . . . , L*ₙ} on each of the subsequent frames. The coarse feature maps of each ultrasound image that characterize the location of the tumor can be extracted via a backbone network. The fixed-size feature maps, which represent the saliency mask of the tumor, can be extracted via a regional proposal network within the enlarged region of interest (ROI) and resized to a fixed size via ROI alignment. These fixed-size feature maps can be fed into fully connected layers to obtain ROI branches, where the ROI branches with classification 1 denote the candidate ROI {L₂^{C}, L₃^{C}, . . . , Lₙ^{C}}. The fixed-size feature maps of two consistent ultrasound frames can then be fed into the ConvLSTM module to obtain an ROI adjustment {δ₂^{C}, δ₃^{C}, . . . , δₙ^{C}} for the previous candidate ROI. Finally, the tracked tumor or tissue ROI {L*₂, L*₃, . . . , L*ₙ} can be obtained via L*ᵢ=Lᵢ^{c}+δᵢ.

FIG. 5B shows an illustrative example of a real-time tracking operation. In FIG. 5B, pane a1 shows the ultrasonic sequence's first frame having a ground truth marker 502. Panes a2-a3 and b1-b3 include five sequential frames showing. Boxes 504 represent the predicted landmark positions in the two different landmark tracking, respectively.

### Inverse Kinematics Analysis

FIG. 2A, diagram 218 shows an example robot's coordinate frames that can be assigned to both the fixed base (e.g., 206) and the moving platform (e.g., 208, 210). In the diagram, the fixed frame B (e.g., 206) of the robot is assigned at the center point on the fixed base at O_{B}, and the moving frame P (e.g., 208, 210) of the robot (e.g., 200, 300) is assigned at the center point on the moving platform at O_{P}. The positions of the universal and spherical joints in frames B and P, respectively, may be expressed per Equations 11-14.

$${}^{p} {p}_{i} = {{r}_{P} [ \begin{matrix}cos ( {γ}_{i} ) & sin ( {γ}_{i} ) & 0\end{matrix} ]}^{T} \qquad ( Eq . 11 )$$

$${}^{B} {b}_{i} = {{r}_{B} [ \begin{matrix}cos ( {Γ}_{i} ) & sin ( {Γ}_{i} ) & 0\end{matrix} ]}^{T} \qquad ( Eq . 12 )$$

$${Γ}_{i} = \frac{( i - 1 ) π}{3} - {α}_{B} ; {γ}_{i} = \frac{( i - 1 ) π}{3} - {α}_{P} , for i = 1 , 3 , 5 \qquad ( Eq . 13 )$$

$${Γ}_{i} = {Γ}_{i - 1} + 2 {α}_{B} ; {γ}_{i} = {γ}_{i - 1} + 2 {α}_{B} , for i = 2 , 4 , 6 \qquad ( Eq . 14 )$$

In Equations 11-21, r_{B} and r_{P} are the radius of the base and the radius of the platform, respectively, and α_{B} and α_{P} are half the angle between the base joint attachment points and platform joint attachment points, respectively.

The inverse kinematics of the robot can solve for the actuator lengths needed to achieve a desired end-effector pose. Diagram 218 shows the vector diagram to solve the inverse kinematics. The translation of the moving platform defined in the fixed frame, ^{B}d, may be determined per Equation 15.

$${}^{B} d = {[ \begin{matrix}x & y & z\end{matrix} ]}^{T} \qquad ( Eq . 15 )$$

The orientation of the moving platform may be described using the three Euler angles: Roll (φ), Pitch (θ), and Yaw (ψ). These angles can be described by a series of rotations with respect to the fixed frame given by the rotation matrix, ^{B}Rₚ, per Equation 16.

$${}^{B} {R}_{p} = [ \begin{matrix}c ψ c θ & c ψ s φ s θ - c φ s ψ & s φ s ψ + c φ c ψ s θ \\ c θ s ψ & c φ c ψ + s φ s ψ s θ & c φ s ψ s θ - c ψ s φ \\ - s θ & c θ s φ & c φ c θ\end{matrix} ] \qquad ( Eq . 16 )$$

In Equation 16, the symbol s and c denote the sine and cosine functions, respectively. The vector loop closure equation for each i^{th} actuator may then be defined per Equation 17.

$${I}_{i} = {}^{B} d + {}^{B} {R}_{p} \cdot {}^{p} {p}_{i} - {}^{B} {b}_{i} \qquad ( Eq . 17 )$$

Using the Euclidean norm on the vector loop equation in Equation 17, the length of the i^{th} actuator may be defined per Equation 18.

$${I}_{i} = 〚 {}^{B} d + {}^{B} {R}_{p} \cdot {}^{p} {p}_{i} - {}^{B} {b}_{i} 〛 \qquad ( Eq . 18 )$$

Needle Alignment. In the clinical workflow, the desired needle target P_{target} and desired point of entry P_{entry} may be determined by the clinician using pre-operative scans. Using image registration, these points can be found with respect to the robot's fixed frame.

Using a straight line motion of the needle, the needle vector axis can be defined by per Equation 19.

$$N = {P}_{target} - {P}_{entry} \qquad ( Eq . 19 )$$

Using N=(Nx, Ny, Nz), the Euler angles required to achieve the desired needle axis vector can be determined per Equations 20-22.

$$φ = {sin}^{- 1} ( \frac{{N}_{y}}{\sqrt{{N}_{y}^{2} + {N}_{z}^{2}}} ) \qquad ( Eq . 20 )$$

$$θ = {sin}^{- 1} ( \frac{{N}_{x}}{\sqrt{{N}_{x}^{2} + {N}_{z}^{2}}} ) \qquad ( Eq . 21 )$$

$$ψ = 0 \qquad ( Eq . 22 )$$

The rotation ψ about the z_{B}-axis may be set to zero to give no additional consideration for the needle's axial rotation. Since the needle insertion module is rigidly fixed to the center of the moving platform, these angles can then directly be set as inputs to the inverse kinematics in Equation 8.

In this alignment application, the translation of the robot along the z-axis may be assumed to be a constant value, h. Using this, the x and y translations of the moving platform (e.g., 210) to be used to align with the needle axis vector can be determined per Equations 23 and 24.

$$x = h sin ( θ ) - {P}_{{entry}_{x}} \qquad ( Eq . 23 )$$

$$y = h sin ( θ ) - {P}_{{entry}_{y}} \qquad ( Eq . 24 )$$

In Equations 23 and 24, where P_{entry}_{ₓ} and P_{entry}_{_{y}} are the x and y locations of the desired entry point, respectively. Lastly, the insertion depth of the needle, l, can be solved for by finding the Euclidean distance between the desired target and the desired entry point per Equation 25.

$$l = ‖ {P}_{target} - {P}_{entry} ‖ \qquad ( Eq . 25 )$$

### Experimental Results and Additional Examples

A study was conducted to design, fabricate, model, and evaluate the exemplary robotic instrument as a parallel robot and a corresponding respiration motion compensation protocol (RMCP) for effective robot-assisted abdominal RFA needle placement. The robot included a Stewart platform with a friction drive roller insertion module for autonomous needle deployment. The respiration motion of a liver is compensated using the respiration sensing pad without the need for continuous radiation exposure of a CT scanner. The study systematically validated the prototyped robot through a number of experiments, including a force modeling experiment of the needle insertion module, a free-space accuracy characterization, a dynamic benchtop targeting accuracy experiment, and a fluoroscopy-guided animal study.

The free-space accuracy characterization experiments indicated that the robotic platform is able to provide a needle tip position and orientation accuracy of 2.00±0.75 mm and 0.81±0.48°, respectively. A dynamic targeting experiment using an ex-vivo liver indicates an improvement in position and orientation error of 57% and 30%, respectively, when using the proposed RMCP. Finally, an animal study using a sexually-mature swine undergoing assisted respiration at nine breaths per minute indicates a 77% reduction in additional insertion displacement when using the RMCP.

In the free-space accuracy characterization, the robot was shown to have a mean positional and orientation error of 2.00±0.75 mm and 0.81±0.48°, respectively, with an FRE of 0.96 mm. The primary source of error is likely due to the linear actuators. Actuonix Motion Devices documents the mechanical repeatability as ±0.2 mm and the backlash of the device as 0.2 mm, potentially producing up to a 0.7 mm and 0.25° error at the needle tip. In the dynamic liver experiments, the robot was shown to have a mean positional error of 2.98±1.26 mm and an orientation error of 3.08±1.07° in the RMCP case, and a mean positional error of 4.43±2.80 mm and an orientation error of 3.58±1.69° in the uncompensated case, with an FRE of 0.92 mm. However, this error is exacerbated with insertion depth in the uncompensated case, where mean positional and orientation error in the RMCP case is 3.79±0.93 mm and 4.34±0.72°, respectively, while in the uncompensated case the mean positional and orientation.

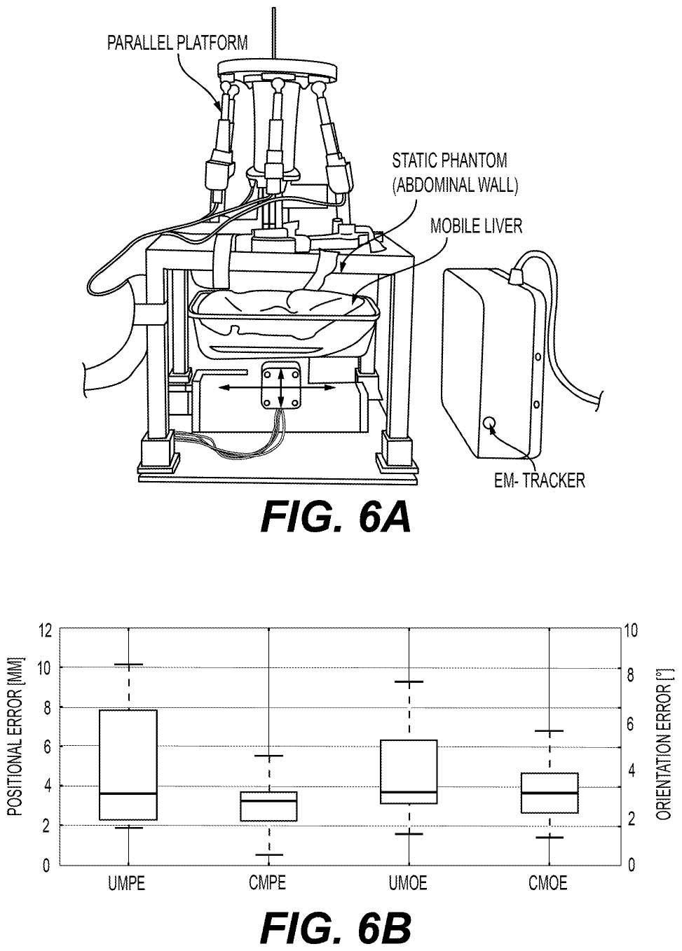

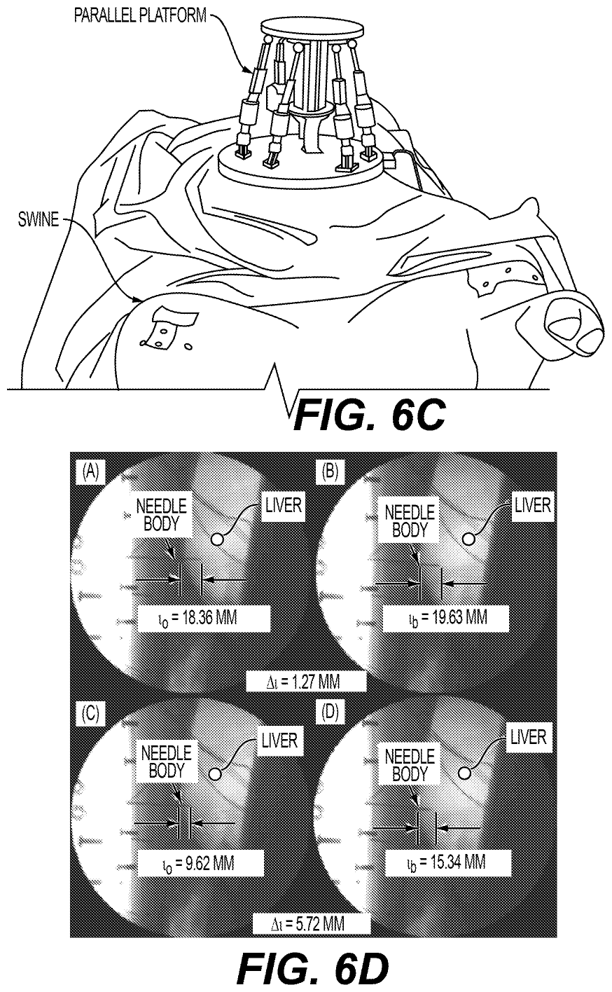

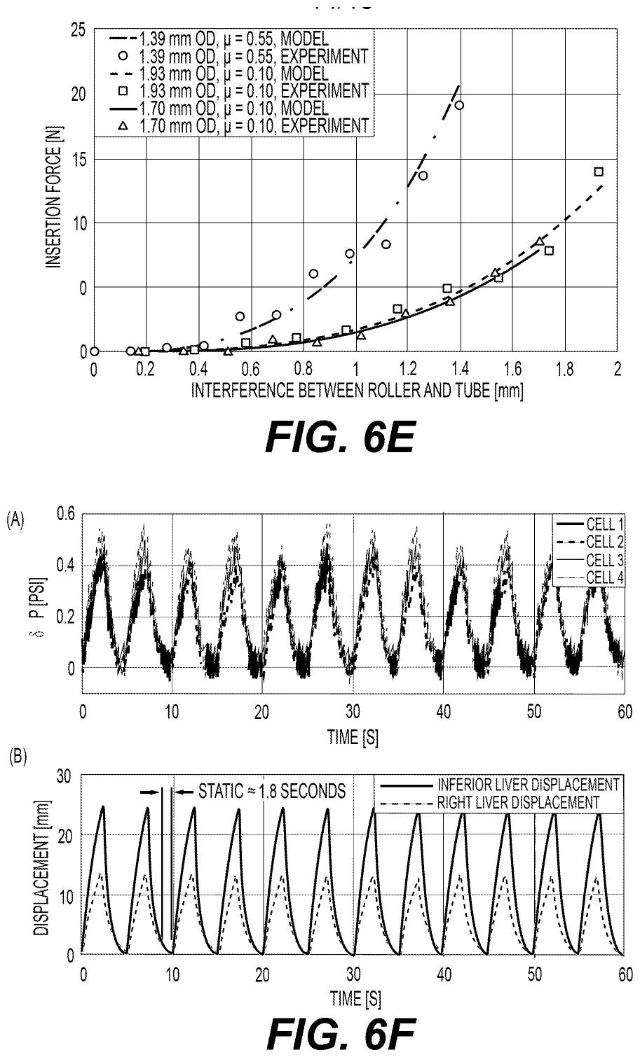

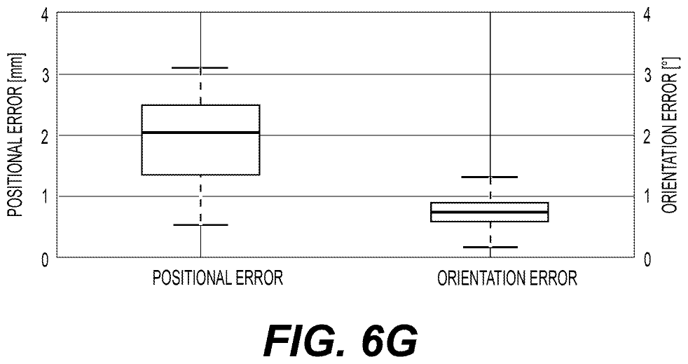

Dynamic Liver Accuracy Characterization. The study evaluated the robot targeting accuracy in a dynamic liver study. FIG. 6A shows an experimental setup for the dynamic ex-vivo study. The arrows indicate the directions of motion of the linear rails.

The study excised liver fresh. To emulate the real procedure, the study moved the liver using a Cartesian platform consisting of two perpendicular lead screws. The base lead screw (inferior-superior (I-S) motion) of the platform was fixed to the ground, and the second lead screw (right-left (R-L) motion) was mounted to a base lead screw's linear table. The study selected a desired motion of these motors to emulate the respiration motion of the liver. In between the liver and the robot, the study placed a static phantom, made of a 5.0% by volume agar/water mixture, between the liver and robot platform. The phantom was approximately 2.5 cm thick, representing the thickness of the abdominal wall. The study then positioned the prototyped robot on top of an aluminum frame fixed relative to the base lead screw and provided the offset necessary between the robot and the liver. The study recorded the needle tip position and orientation using an Aurora EM tracking system (NDI Medical, Waterloo, ON, Canada). The study placed a 5-DoF EM tracking sensor (NDI Medical, Waterloo, ON, Canada) at the needle tip using a thin layer of heat shrink.

The study selected forty desired targets within the robot's workspace to evaluate the robot's targeting performance. Twenty points were reached using the RMCP, and 20 points were uncompensated. These points were divided into four primary groups based on insertion depth from the surface of the static agar phantom. These depths were 30, 45, 60, and 75 mm. Recall that the agar phantom thickness was 2.5 cm; additionally, note the total possible depth before reaching the bottom of the liver container was 80 mm. Each group of four had five insertion attempts, with each attempt increasing the needle orientation angle by 2° (i.e., 0°, 2°, 4°, 6°, 8°). Each attempt was given a different entry point, ensuring previous trajectories were not traversed. Additionally, the static phantom was replaced with a new phantom between the RMCP and uncompensated experiments.

The insertion procedure was performed as follows. First, a desired target and entry point were selected based on relative liver motion positions of zero in both the I-S and R-L coordinates. The inverse kinematics was then used to provide the desired needle axis vector, and the robot was moved to that position. The initial EM tracking sensor position was then recorded. The liver motion and needle insertion were then initiated, starting from an initial relative liver motion position of zero in both the I-S and R-L coordinates. In the RMCP case, changes in pressure readings corresponding to the associated change in motion were relayed to the robotic system, informing the robot when to perform needle insertion. This protocol was followed until the final insertion depth was reached. In the respiratory motion uncompensated case, the robot was allowed to insert the needle continuously until the desired insertion depth was reached. All power was then disabled at the relative liver motion positions of zero in both the I-S and R-L coordinate. This (i) reduced signal noise in the EM-tracking system and (ii) ensured the target position relative to the liver was in the same position relative to the robot frame at the start of the experiment. The final position of the EM-tracking coil was then recorded.

FIG. 6B shows a comparison of positional error (left) for the uncompensated motion (UMPE) and compensated motion (CMPE) case and orientation error (right) for the uncompensated motion (UMOE) and compensated motion (CMOE) case of the dynamic ex-vivo targeting experiment.

The results of the dynamic targeting test indicate a mean positional error of 2.98\textpm1.26 mm and an orientation error of 3.08±1.07° in the RMCP case, and a mean positional error of 4.43±2.80 mm and an orientation error of 3.58±1.69° in the uncompensated case, with an FRE of 0.92 mm. With increasing insertion depth, it was observed that the error did not significantly change in the RMCP case; however, for the uncompensated case, the error increased significantly. For example, at an insertion depth of 75 mm, the mean positional and orientation error in the compensated case are 3.79±0.93 mm and 4.34±0.72°, respectively, while in the uncompensated case, the mean positional and orientation error are 8.93±0.79 mm and 6.17±0.92°, respectively.

Needle & Roller Force Modeling. The study employed strain energy models to predict needle insertion force. The study characterized the force modeling of the friction drive roller insertion module by mounting a modified version of the roller insertion module at the end of a stepper motor (NEMA 23) driven lead screw. This version of the insertion module had rollers that did not rotate, emulating the condition when the driving rollers were held stationary by the enabled motor.

The rollers of this version could be moved laterally with respect to the needle body, allowing different interference gaps for testing. The study set the interference gap using feeler gauges with known thicknesses. The study mounted a force sensor (Go Direct Force and Acceleration Sensor, Vernier, USA) to the linear table, and the needles used for validation were attached to the sensor at the proximal end of the needle. The study used three different needles: (i) a standard sheath for breast biopsy made of polished stainless steel with OD 1.90 mm (μ=0.1), (ii) a standard needle for breast biopsy made of polished stainless steel with OD 1.70 mm (μ=0.1), and (iii) a cold-rolled steel rod, with OD 1.39 mm (μ=0.55).

Based on the needle size, the study tested 11 different, equally spaced roller gaps with the first gap being the same as the needle diameter (i.e., insertion force=0). At each gap, the needle was inserted 10 times through the rollers while the force of the insertion was recorded. The study recorded the peak force as the insertion force for the insertion module. FIG. 6E shows a comparison of the force model to the average insertion force for each gap of each needle. A mean error of 0.49±0.28 N was found across the needle insertion experiments. The errors seen can be attributed to inaccuracies in the roller gap and the approximation of the modulus of elasticity. However, the results suggest that the model can successfully be applied to approximate the insertion force of roller-based insertion mechanisms. This model suggests that the needle can successfully puncture a porcine abdominal cavity, with penetration forces ranging in the 0.81 to 4.2 N range [37]. To validate this, each needle was successfully inserted into a skin-on porcine belly sample, easily overcoming the tissue elasticity using the roller design [38].

Animal Study. The study evaluated the RMCP efficacy in an animal study at the Global Center for Medical Innovation T3 Labs with Institutional IACUC approval with a sexually mature (>6mth) female swine. The study positioned the swine in the supine position on an operating table next to a C-arm.

FIG. 6C shows the experimental setup for the swine-based experiment. The C-Arm was used to provide visual feedback of the needle's motion during respiration. The swine remained under anesthesia during the procedure and was given a respiration rate of nine breaths per minute. The study positioned the robot on the swine so that the liver was in the robot's workspace. The robot was then strapped to the swine using standard nylon straps. Due to the absence of 3D-registration, the study manually placed the needle in the liver while respiration was paused without targeting consideration. The study then recorded the needle position using fluoroscopy during respiration while RMCP was engaged (i.e., the insertion module did not grasp the needle, which permitted needle motion with respiration). FIG. 6G shows the results of the needle motion with compensation. The study then repositioned the needle and the needle position was again recorded using fluoroscopy during respiration while RMCP was disengaged (i.e. insertion module grasped the needle, which restricted needle motion with respiration). FIG. 6D shows the results of the needle motion without compensation. In Pane A, FIG. 6D shows the released needle during complete exhalation; Pane B shows the released needle during complete inhalation; Pane C shows the grasped needle during complete exhalation; and Pane D shows the grasped needle during complete inhalation.

Respiration to Pressure Mapping. The purpose of this section was to create a mapping between respiration and pressure readings in the respiration sensing pad. To generate this mapping, the linear actuators, upper platform, and insertion module was removed from the lower platform. The respiration sensing pad remained on the bottom platform, and the pair were then strapped to a volunteer. The volunteer was permitted to respire naturally for 60 seconds while the change in pressure readings in the four cells was recorded in real-time. These results can be seen in FIG. 8A, where the peaks represent maximum inhalation and the troughs indicate maximum exhalation. Based on these results, when the change in pressure is less than 0.10 psi, the robot should insert the needle. Once the change in pressure is greater than 0.10 psi, the robot should release the needle, engaging the RMCP. This permits approximately 1.8 seconds per cycle for needle insertion.

The study used this data to provide a desired trajectory for the Cartesian platform to follow, emulating the dynamic liver motion in the Dynamic Liver Accuracy Characterization evaluation. To obtain this information, the study averaged the change in pressure data between the four cells and then normalized them between the maximum and minimum average change in pressure on a scale of zero to one. An 8th-order Fourier approximation was then fit to the normalized data, as the respiration cycle is periodic with a frequency of approximately 0.2 Hz. The study then multiplied the normalized approximated data multiplied by the maximum anticipated liver displacements in the inferior (24.4 mm) and right (13.2 mm) directions [10]. This provided the desired motion of the Cartesian platform for emulating the liver motion (FIG. 6F, lower pane). FIG. 6F, top pane shows pressure data recorded using the pressure sensing array, while the bottom pane shows displacement position commands sent to the stepper motors for the dynamic ex-vivo liver test . . .

Free Space Accuracy Validation of the System. The robot free space targeting accuracy was evaluated using an electromagnetic (EM) tracking system (NDI Medical, Waterloo, ON, Canada). The robot was positioned on top of an aluminum frame, placing the needle tip in the middle of the EM-tracker workspace. Needle tip position and orientation were recorded using an Aurora 5-DoF EM tracking sensor (NDI Medical, Waterloo, ON, Canada). The sensor was fixed to the needle tip using a thin layer of heat-shrink. Coordinate registration was performed to convert the sensor's coordinate frame to the robot coordinate frame [26].

A total of fifty desired targets (needle tip positions) were selected within the robot's workspace to evaluate the robot's free space accuracy. The 50 target positions were divided into groups of five. Each group of five had a different desired (x,y) needle tip position in the robot frame. These included (5,0), (0,5), (−5, 0), (0,−5), (10,0), (0,10), (−10, 0), (0,−10), (20,0), (0,20). Within each group of five, five different needle orientation angles were targeted for (ϕ,θ) in degrees. These were (0,0), (8,0), (0,8), (−8,0), and (0,−8), which were the maximum possible angles available to all needle tip positions. The mean positional error was 2.00±0.75 mm and the mean orientation error was 0.81±0.48°, with an FRE of 0.96 mm. FIG. 6G shows the results of this experiment.

The primary source of error is likely attributed to the linear actuators. Actuonix Motion Devices documents the mechanical repeatability ±0.2 mm and the backlash of the device as 0.2 mm. In the worst-case scenario, this could result in opposing actuator pairs (i.e., 1, 2 to 4, 5) having a difference between each other of 0.4 mm. Due to the offset of the needle tip from the upper platform of 165 mm, this can result in a total positional and orientation error of the needle tip of ±0.72 mm and ±0.25°.

### Discussion

Table 1 shows a comparison of various robotic platforms. It is the inventor's knowledge that the exemplary robotic needle insertion system is the first body-mounted system to provide robotic stepwise needle insertion operation. A predecessor system is disclosed in [41], which is incorporated herein.

TABLE 1

| | | | Tumor | | |
|---|---|---|---|---|---|
| Robot | Mounting | | position | Targeting | |
| platform | method | Needle insertion | update | error | Ref. |
| ROBIO | Floor-mounted | Stepwise (manual) | No | N/A | [34′] |
| Double-ring | Body-mounted | Stepwise (manual) | No | 6 mm | [35′] |
| RCM | Floor-mounted | Stepwise (manual) | No | 1.0 ± 0.4 mm | [36′] |
| Hybrid | Gantry-mounted | Stepwise (manual) | No | <5 mm | [37′] |
| robot | | | | | |
| DLR/KUKA | Floor-mounted | Stepwise (manual) | No | 1.2 ± 0.4 mm | [38′] |
| Franco robot | Gantry-mounted | Stepwise (manual) | No | 2.9 ± 1.4 mm | [39′] |
| Shoulder | Body-mounted | Stepwise (manual) | No | 3.4 ± 1.65 mm | [40′] |
| robot | | | | | |
| Pain robot | Body-mounted | Stepwise (manual) | No | 1.7 ± 0.21 mm | [41′] |
| Arches | Table-mounted | Continuous | No | <10 mm | [42′] |
| robot | | (robotic) | | | |
| AcuBot | Gantry-mounted | Continuous | No | 1.44 ± 0.66 mm | [43′] |
| | | (robotic) | | | |
| Robopsy | Body-mounted | Continuous | No | 4.3 ± 1.6 mm | [44′] |
| | | (robotic) | | | |
| Exemplary | Body-mounted | Continuous | Yes | ±0.72 mm | |
| robot | | Stepwise (robotic) | | and ±0.25° | |

It is noted that the body-mounted approach could passively compensate for the respiration motion [11′]. It is noted that the continuous stepwise needle insertion could ‘intelligently’ gate the respiration motion, a.k.a. the clinician typically inserts the needle when the tumor motion is minimal and releases the needle when the motion is maximum. It is noted that the real-time tumor position update provides critical operation to guide the procedure. The exemplary robotic instrument can provide all three functions simultaneously.

Cancer Discussion. Cancer is the leading cause of death in the world, accounting for nearly 10 million deaths in 2020 [1]. Of these, lung and breast cancer account for the majority of deaths, with a projected 130,180 and 43,780 deaths in 2022, respectively; however, other cancer-related deaths, such as liver cancer, are on the rise, nearly doubling since 1980 [2]. This has motivated clinicians to explore a variety of liver cancer treatments. Treatments can be divided into four major categories: surgical, systemic therapy, radiotherapy, and thermal therapy [3].

Radiofrequency Ablation (RFA), a subgroup of thermal therapy, is currently considered an effective treatment method for cancer treatment, which relies on low-frequency wavelengths to generate heat within a tumor, causing thermal coagulative necrosis [4]. RFA is typically performed percutaneously and was first used as a treatment for patients with Hepatocellular carcinoma (HCC) excluded from surgical resection [5]. The advantages of RFA are well documented for HCC and include the following (i) minimally invasive, enabling outpatient procedures, (ii) safer approach, improving morbidity and mortality, (iii) excellent ablation energy localization, (iv) comparable long term survival rates to resection, and (v) excellent therapeutic candidate for multimodal treatment [6].

Despite the many advantages of RFA, there are several limitations that adversely affect clinical efficacy, the primary of which is the ablation needle placement accuracy. State-of-the-art leverages intraprocedural image guidance (ultrasound [7], [8], MRI [9], or CT [4]) to localize the ablation needle with respect to the target. However, manually placing the needle at a desired location remains a prevalent clinical challenge due to respiration-induced movement [10]. This movement can be large, being as much as 24 mm in the liver [10], [11]. To address this issue, breath-holding is typically required during needle placement to reduce organ motion, which can be difficult for patients due to the significant pain associated with RFA procedures [12].

Several groups have investigated robotic solutions that aid in percutaneous needle-based interventions [13], [14]. These can primarily be divided into two major categories: 1) floor-mounted systems (i.e. mounted to the floor, scanner, ceiling, etc.) and 2) patient-mounted systems. Baere et al. [15] used the commercially available EPIONE® robotic device (Quantum Surgical, Montpellier, France) with respiratory monitoring for CT-guided percutaneous needle insertion in the kidney. Zhou et al. implemented a Mitsubishi RV-E2 general-purpose 6-DOF articulated robot manipulator for CT-guided needle biopsy of lung nodules for lung cancer; needle advancement was gated using CT feedback. Stoianovici et al. developed AcuBot, a CT scanner-mounted robot for percutaneous radiological interventions. Duan et al. developed a rail-mounted robotic RCM mechanism on top of a two DoF linear slide for RFA of large liver tumors. However, these floor-mounted systems are not able to compensate for any unexpected patient motion during the procedure.

Additionally, these robots are inherently large and bulky, which can increase the preparation time and the procedure time. Patient-mounted robots have the advantage of being smaller since they do not require special support bases or frames, such as [19], [20], [21]. The compact dimension of patient-mounted robot allows passive motion compensation by allowing robot movement with the body, reducing complexity. Despite this inherent benefit, these robots can still miss the target in practical applications due to the inherent motion of abdominal organs, such as the liver, with respect to the skin. For example, the double-ring robot reported in [21] could achieve a targeting accuracy of approximately 6 mm in phantoms, but maintaining this accuracy would be difficult when attempting to reach a dynamic target in liver-based applications, which has an average motion of 13.2±6.9 mm, 24.4±16.4 mm, and 9.0±3.5 mm in the left-right, cranial-caudal, and anterior-posterior directions, respectively [11].

In contrast, the exemplary robotic platform employs a patient-mounted, respiratory motion-compensated robotic system for accurate RFA needle placement. The exemplary robotic platform can be employed as a dexterous robotic platform for RFA needle delivery, among other applications described herein. The platform was optimized using a force modeling method for friction drive roller mechanisms used in needle insertion based on strain energy methods.

Liver cancer discussion. Liver cancer, also known as hepatocellular carcinoma (HCC), is a significant human health challenge. In the United States, an estimated 42,000 patients will be diagnosed each year, leading to more than 30,000 deaths (the five-year survival rate of HCC ranges between 3% and 34%) [2]. The number of cases is expected to rise continuously due to the increasing number of chronic liver diseases caused by alcohol, nonalcoholic fatty liver disease, hepatitis B, and hepatitis C infection. The annual incremental medical cost of an HCC patient ranges from $50,000 to $500,000, which leads to more than one billion costs per year [3]. Due to its high prevalence, substantial medical expenses, and high mortality rate, curing liver cancer presents challenges to governments, clinicians, and patients.

Limitations of conventional treatments. HCC can be treated with a variety of methods depending on tumor size, whether the tumor has spread, and underlying damage to the liver tissue [4]-[10]. In current practice, percutaneous ablation (energy source can be radiofrequency, laser, or microwave) is the primary option for tumors less than 5 cm. The main advantages of ablation include 1) minimally invasive, 2) its capability to enable focal tumor control, 3) its favorable long-term survival rate, and 4) it can be combined with other treatment approaches [10]. A recent study suggested that “it could be locally curative for HCC and might be the first-line treatment for selected patients with early-stage HCC” [11]. Despite these promising benefits, it does present some clinical challenges. Due to the limited ablation volume and heat shrink effect within the liver tissue, the ablation needle has to be accurately placed within the tumor to create the desired coagulation zone to encompass the entire target. However, manually placing the ablation needle into the dynamic tumor has been a long-standing challenge, even with intra-procedural image guidance (see FIG. 1). Needle targeting error is mainly caused by the respiration-induced movement of the liver tumor, which can be as large as 5.5 cm in the superior-inferior direction [13], [14]. In addition to suboptimal or incomplete treatment, inaccurate needle placement could also lead to undesired collateral thermal injury, especially when the tumor is located close to the colon, diaphragm, gallbladder, or main bile duct [10].

To address this problem, breath-holding is typically required during needle placement to mitigate respiration-induced motion. However, it can be difficult for the patients to hold their breath due to compromised lung capability, the intravenous sedation administered during the procedure, and the significant pain induced by ablation during the procedure. Thus, manually deploying the ablation needle with breath-holding, especially for those targets located in the upper portion of the liver, can be a challenging task in clinical settings [15]. Also, manual insertion for HCC treatment is a crucial step that relies heavily on the clinician's experience and may lead to variations in surgical outcomes [16]. Many research groups have developed robotic platforms to assist in needle insertion during percutaneous procedures. Review articles on these robotic devices can be found in Kettenbach et al. [17] and Arnolli et al. [18]. These robots have shown clinical promise; however, none of them have addressed respiration-induced organ motion as most of the accuracy tests were performed in a static phantom or cadaver. An accurate needle insertion system that overcomes the limitations associated with the manual approach and considers more practical factors may allow for improved care for these patients [15].

In contrast, the exemplary robotic needle insertion system can improve the treatment for HCC as well as metastatic liver tumors via superior ablation needle targeting. The robotic hardware and navigation software framework can also be helpful for other cancer diagnoses and treatments. For example, the exemplary robotic needle insertion system could be potentially used for lung cancer biopsy. Lung cancer kills over 130,000 people in America each year. Early diagnosis is critical for optimal treatment. Stage-I lung cancer patients have a 10-year survival rate of 88%, while stage III or IV has only a 15% 5-year survival rate. Biopsy procedures are required to obtain a definitive diagnosis of suspicious nodules [27]. A system that can precisely deploy the biopsy needle towards the target of interest could lead to accurate diagnosis for lung cancers, resulting in increased survival of these patients. The exemplary robotic needle insertion system can be used for draining systems, among other applications.

### Example Computing System

It should be appreciated that the logical operations described above can be implemented (1) as a sequence of computer-implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts, and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

The computer system is capable of executing the software components described herein for the exemplary method or systems. In an embodiment, the computing device may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computing device to provide the functionality of a number of servers that are not directly bound to the number of computers in the computing device. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or can be hired on an as-needed basis from a third-party provider. Some cloud computing environments may comprise cloud computing resources owned and operated by the enterprise as well as cloud computing resources hired and/or leased from a third-party provider.

In its most basic configuration, a computing device includes at least one processing unit and system memory. Depending on the exact configuration and type of computing device, system memory may be volatile (such as random-access memory (RAM)), non-volatile (such as read-only memory (ROM), flash memory, etc.), or some combination of the two.

The processing unit may be a standard programmable processor that performs arithmetic and logic operations necessary for the operation of the computing device. While only one processing unit is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application-specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device.

Computing devices may have additional features/functionality. For example, the computing device may include additional storage, such as removable storage and non-removable storage, including, but not limited to, magnetic or optical disks or tapes. Computing devices may also contain network connection(s) that allow the device to communicate with other devices, such as over the communication pathways described herein. The network connection(s) may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing devices may also have input devices(s) such as keyboards, keypads, switches, dials, mice, trackballs, touch screens, voice recognizers, card readers, paper tape readers, or other well-known input devices. Output device(s) such as printers, video monitors, liquid crystal displays (LCDs), touch screen displays, displays, speakers, etc., may also be included. The additional devices may be connected to the bus in order to facilitate the communication of data among the components of the computing device. All these devices are well-known in the art and need not be discussed at length here.

The processing unit may be configured to execute program code encoded in tangible, computer-readable media. Tangible, computer-readable media refers to any media that is capable of providing data that causes the computing device (i.e., a machine) to operate in a particular fashion. Various computer-readable media may be utilized to provide instructions to the processing unit for execution. Example tangible, computer-readable media may include but is not limited to volatile media, non-volatile media, removable media, and non-removable media implemented in any method or technology for storage of information such as computer-readable instructions, data structures, program modules, or other data.

In light of the above, it should be appreciated that many types of physical transformations take place in the computer architecture in order to store and execute the software components presented herein. It also should be appreciated that the computer architecture may include other types of computing devices, including hand-held computers, embedded computer systems, personal digital assistants, and other types of computing devices known to those skilled in the art.

In an example implementation, the processing unit may execute program code stored in the system memory. For example, the bus may carry data to the system memory, from which the processing unit receives and executes instructions. The data received by the system memory may optionally be stored on the removable storage or the non-removable storage before or after execution by the processing unit.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language, and it may be combined with hardware implementations.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entirety and to the same extent as if each reference was individually incorporated by reference.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

As discussed herein, a “subject” may be any applicable human, animal, or other organism, living or dead, or other biological or molecular structure or chemical environment, and may relate to particular components of the subject, for instance, specific tissues or fluids of a subject (e.g., human tissue in a particular area of the body of a living subject), which may be in a particular location of the subject, referred to herein as an “area of interest” or a “region of interest.”

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications, and publications, as listed below and throughout this document, are hereby incorporated by reference in their entirety herein.

### REFERENCE LIST #1

[1] World Health Organization (WHO), “Cancer,” 2022. https://www.who.int/news-room/fact-sheets/detail/cancer, Last accessed on 2022 Aug. 27.
[2] American Cancer Society, “Cancer facts & figures 2022,” 2022. https://www.cancer.org/research/cancer-facts-statistics/all-cancer-factsfigures/cancer-facts-figures-2022.html, Last accessed on 2022 Aug. 27.
[3] J. Balogh, D. Victor III, E. H. Asham, S. G. Burroughs, M. Boktour, A. Saharia, X. Li, R. M. Ghobrial, and H. P. Monsour Jr, “Hepatocellular carcinoma: a review,” Journal of hepatocellular carcinoma, vol. 3, p. 41, 2016.
[4] D. R. Shah, S. Green, A. Elliot, J. P. McGahan, and V. P. Khatri, “Current oncologic applications of radiofrequency ablation therapies,” World journal of gastrointestinal oncology, vol. 5, no. 4, p. 71, 2013.
[5] S. A. Curley, F. Izzo, L. M. Ellis, J. N. Vauthey, and P. Vallone, “Radiofrequency ablation of hepatocellular cancer in 110 patients with cirrhosis,” Annals of surgery, vol. 232, no. 3, p. 381, 2000.
[6] H. Rhim and H. K. Lim, “Radiofrequency ablation of hepatocellular carcinoma: pros and cons,” Gut and liver, vol. 4, no. Suppl 1, p. S113, 2010.
[7] X. Dai, Y. Lei, J. Roper, Y. Chen, J. D. Bradley, W. J. Curran, T. Liu, and X. Yang, “Deep learning-based motion tracking using ultrasound images,” Medical Physics, vol. 48, no. 12, pp. 7747-7756, 2021.
[8] Y. Zhang, X. Dai, Z. Tian, Y. Lei, Y. Chen, P. Patel, J. D. Bradley, T. Liu, and X. Yang, “Liver motion tracking in ultrasound images using attention guided mask r-cnn with long-short-term-memory network,” in Medical Imaging 2022: Ultrasonic Imaging and Tomography, vol. 12038, pp. 156-161, SPIE, 2022.
[9] L. Boldrini, A. Romano, S. Mariani, D. Cusumano, F. Catucci, L. Placidi, G. C. Mattiucci, G. Chiloiro, F. Cellini, M. A. Gambacorta, et al., “Mri-guided stereotactic radiation therapy for hepatocellular carcinoma: a feasible and safe innovative treatment approach,” Journal of Cancer Research and Clinical Oncology, vol. 147, no. 7, pp. 2057-2068, 2021.
[10] B. Bussels, L. Goethals, M. Feron, D. Bielen, S. Dymarkowski, P. Suetens, and K. Haustermans, “Respiration-induced movement of the upper abdominal organs: a pitfall for the three-dimensional conformal radiation treatment of pancreatic cancer,” Radiotherapy and Oncology, vol. 68, no. 1, pp. 69-74, 2003.
[11] I. Suramo, M. Päivänsalo, and V. Myllylä, “Cranio-caudal movements of the liver, pancreas and kidneys in respiration,” Acta radiologica. Diagnosis, vol. 25, no. 2, pp. 129-131, 1984.
[12] R. T. Poon, K. K. Ng, C. M. Lam, V. Ai, J. Yuen, S. T. Fan, and J. Wong, “Learning curve for radiofrequency ablation of liver tumors: prospective analysis of initial 100 patients in a tertiary institution,” Annals of surgery, vol. 239, no. 4, p. 441, 2004.
[13] M. M. Arnolli, N. C. Hanumara, M. Franken, D. M. Brouwer, and I. A. Broeders, “An overview of systems for ct- and mri-guided percutaneous needle placement in the thorax and abdomen,” The International Journal of Medical Robotics and Computer Assisted Surgery, vol. 11, no. 4, pp. 458-475, 2015.
[14] J. Kettenbach and G. Kronreif, “Robotic systems for percutaneous needle-guided interventions,” Minimally Invasive Therapy & Allied Technologies, vol. 24, no. 1, pp. 45-53, 2015.
[15] S. Daher, M. Massarwa, A. A. Benson, and T. Khoury, “Current and future treatment of hepatocellular carcinoma: an updated comprehensive review,” Journal of clinical and translational hepatology, vol. 6, no. 1, p. 69, 2018.
[16] Y. Zhou, K. Thiruvalluvan, L. Krzeminski, W. H. Moore, Z. Xu, and Z. Liang, “An experimental system for robotic needle biopsy of lung nodules with respiratory motion,” in 2011 IEEE International Conference on Mechatronics and Automation, pp. 823-830, IEEE, 2011.
[17] D. Stoianovici, K. Cleary, A. Patriciu, D. Mazilu, A. Stanimir, N. Craciunoiu, V. Watson, and L. Kavoussi, “Acubot: a robot for radiological interventions,” IEEE Transactions on Robotics and Automation, vol. 19, no. 5, pp. 927-930, 2003.
[18] B. Duan, R. Wen, C.-B. Chng, W. Wang, P. Liu, J. Qin, J. L. Peneyra, S. K.-Y. Chang, P.-A. Heng, and C.-K. Chui, “Image-guided robotic system for radiofrequency ablation of large liver tumor with single incision,” in 2015 12th International Conference on Ubiquitous Robots and Ambient Intelligence (URAI), pp. 284-289, IEEE, 2015.
[19] L. Maier-Hein, C. J. Walsh, A. Seitel, N. C. Hanumara, J.-A. Shepard, A. M. Franz, F. Pianka, S. A. Müller, B. Schmied, A. H. Slocum, et al., “Human vs. robot operator error in a needle-based navigation system for percutaneous liver interventions,” in Medical Imaging 2009: Visualization, Image-Guided Procedures, and Modeling, vol. 7261, pp. 314-325, SPIE, 2009.
[20] N. Hungr, C. Fouard, A. Robert, I. Bricault, and P. Cinquin, “Interventional radiology robot for ct and mri guided percutaneous interventions,” in International Conference on Medical Image Computing and Computer-Assisted Intervention, pp. 137-144, Springer, 2011.
[21] N. Hata, S.-E. Song, O. Olubiyi, Y. Arimitsu, K. Fujimoto, T. Kato, K. Tuncali, S. Tani, and J. Tokuda, “Body-mounted robotic instrument guide for image-guided cryotherapy of renal cancer,” Medical physics, vol. 43, no. 2, pp. 843-853, 2016.
[22] M. J. Musa, K. Sharma, K. Cleary, and Y. Chen, “Respiratory compensated robot for liver cancer treatment: Design, fabrication, and benchtop characterization,” IEEE/ASME Transactions on Mechatronics, vol. 27, no. 1, pp. 268-279, 2021.
[23] D. Stewart, “A platform with six degrees of freedom,” in: Proceedings of institute of mechanical engineering, vol. 180, pp. 371-86, 1965.
[24] M. Musa, S. Sengupta, and Y. Chen, “Design of a 6-dof parallel robotic platform for mri applications,” Journal of Medical Robotics Research, vol. 0, no. 0, p. 2241005, 0.
[25] M. Musa, S. Sengupta, and Y. Chen, “Mri-compatible soft robotic sensing pad for head motion detection,” IEEE Robotics and Automation Letters, vol. 7, no. 2, pp. 3632-3639, 2022.
[26] J. M. Fitzpatrick, J. B. West, and C. R. Maurer, “Predicting error in rigidbody point-based registration,” IEEE transactions on medical imaging, vol. 17, no. 5, pp. 694-702, 1998.
[27] R. J. Webster, J. Memisevic, and A. M. Okamura, “Design considerations for robotic needle steering,” in Proceedings of the 2005 IEEE International Conference on Robotics and Automation, pp. 3588-3594, IEEE, 2005.
[28] C. J. Walsh, N. C. Hanumara, A. H. Slocum, J.-A. Shepard, and R. Gupta, “A patient-mounted, telerobotic tool for ct-guided percutaneous interventions,” Journal of Medical Devices, vol. 2, no. 1, 2008.
[29] J. Hong, T. Dohi, M. Hashizume, K. Konishi, and N. Hata, “An ultrasound-driven needle-insertion robot for percutaneous cholecystostomy,” Physics in Medicine & Biology, vol. 49, no. 3, p. 441, 2004.
[30] S. Shin, W. Park, H. Cho, S. Park, and L. Kim, “Needle insertion simulator with haptic feedback,” in International Conference on Human-Computer Interaction, pp. 119-124, Springer, 2011.
[31] M. Khadem, C. Rossa, N. Usmani, R. S. Sloboda, and M. Tavakoli, “A two-body rigid/flexible model of needle steering dynamics in soft tissue,” IEEE/ASME Transactions on Mechatronics, vol. 21, no. 5, pp. 2352-2364, 2016.
[32] Y. Chen, A. Squires, R. Seifabadi, S. Xu, H. K. Agarwal, M. Bernardo, P. A. Pinto, P. Choyke, B. Wood, and Z. T. H. Tse, “Robotic system for mri-guided focal laser ablation in the prostate,” IEEE/ASME Transactions on Mechatronics, vol. 22, no. 1, pp. 107-114, 2016.
[33] A. L. Gunderman, E. J. Schmidt, M. Morcos, J. Tokuda, R. T. Seethamraju, H. R. Halperin, A. N. Viswanathan, and Y. Chen, “Mrtracked deflectable stylet for gynecologic brachytherapy,” IEEE/ASME Transactions on Mechatronics, vol. 27, no. 1, pp. 407-417, 2021.
[34] P. J. Swaney, P. A. York, H. B. Gilbert, J. Burgner-Kahrs, and R. J. Webster, “Design, fabrication, and testing of a needle-sized wrist for surgical instruments,” Journal of medical devices, vol. 11, no. 1, 2017.
[35] F. Beer, E. Johnston, J. DeWolf, and D. Mazurek, “Mechanics of materials. 7th_edition,” New York. MeGraw-Hill Education Ltd, 2015.
[36] K. Larson, “Can you estimate modulus from durometer hardness for silicones,” Dow Corning Corporation, pp. 1-6, 2016.
[37] S. Cronin, F. I. Mary, and A. Mathew, “Puncture force measurements on a porcine stomach,” Gastrointestinal Endoscopy, vol. 65, no. 5, p. AB294, 2007.
[38] X. Bao, W. Li, M. Lu, and Z. Zhou, “Experiment study on puncture force between mis suture needle and soft tissue,” Biosurface and Biotribology, vol. 2, no. 2, pp. 49-58, 2016.
[39] M. Earle, G. De Portu, and E. DeVos, “Agar ultrasound phantoms for low-cost training without refrigeration,” African Journal of Emergency Medicine, vol. 6, no. 1, pp. 18-23, 2016.
[40] S. Su, W. Wang, D. Nadebaum, A. Nicoll, S. Sood, A. Gorelik, J. Lai, and R. Gibson, “Skin-liver distance and interquartile range-median ratio as determinants of interoperator concordance in acoustic radiation force impulse imaging,” Journal of Medical Ultrasound, vol. 27, no. 4, p. 177, 2019.
[41] US20220142702A1

### REFERENCE LIST #2

[1′] W. Wein, S. Brunke, A. Khamene, M. R. Callstrom, and N. Navab, “Automatic CT-ultrasound registration for diagnostic imaging and image-guided intervention,” Med. Image Anal., vol. 12, no. 5, pp. 577-585, 2008.
[2′] H. Kim and H. B. El-Serag, “The Epidemiology of Hepatocellular Carcinoma in the USA,” Curr. Gastroenterol. Rep., vol. 21, no. 4, p. 17, 2019.
[3′] L. G. Mantovani and M. Strazzabosco, “Healthcare costs associated with hepatocellular carcinoma and the value of care,” Hepatology, vol. 58, no. 4, pp. 1213-1214, 2013.
[4′] J. M. Llovet et al., “Sorafenib in advanced hepatocellular carcinoma,” N. Engl. J. Med., vol. 359, no. 4, pp. 378-390, 2008.
[5′] S. Ma et al., “Approach to radiation therapy in hepatocellular carcinoma,” Cancer Treat. Rev., vol. 36, no. 2, pp. 157-163, 2010.
[6′] H. Nakamura et al., “Transcatheter embolization of hepatocellular carcinoma: assessment of efficacy in cases of resection following embolization,” Radiology, vol. 147, no. 2, pp. 401-405, 1983.
[7′] V. Mazzaferro et al., “Milan criteria in liver transplantation for hepatocellular carcinoma: an evidence-based analysis of 15 years of experience,” Liver Transplant., vol. 17, no. S2, pp. S44-S57, 2011.
[8′] R. Wong and C. Frenette, “Updates in the management of hepatocellular carcinoma,” Gastroenterol. Hepatol. (N. Y)., vol. 7, no. 1, p. 16, 2011.
[9′] K. Goyal et al., “Cyberknife stereotactic body radiation therapy for nonresectable tumors of the liver: preliminary results,” HPB Surg., vol. 2010, 2010.
[10′] H. Rhim and H. K. Lim, “Radiofrequency ablation of hepatocellular carcinoma: pros and cons,” Gut Liver, vol. 4, no. Suppl 1, p. S113, 2010.
[11′] S. Shiina et al., “Radiofrequency ablation for hepatocellular carcinoma: 10-year outcome and prognostic factors,” Am. J. Gastroenterol., vol. 107, no. 4, p. 569, 2012.
[12′] “Liver Cancer: Statistics.” [Online′]. Available: http://cancer.net/cancer-types/liver-cancer/statistics.
[13′] B. Bussels et al., “Respiration-induced movement of the upper abdominal organs: a pitfall for the three-dimensional conformal radiation treatment of pancreatic cancer,” Radiother. Oncol., vol. 68, no. 1, pp. 69-74, 2003.
[14′] I. Suramo, M. Päivänsalo, and V. Myllylä, “Cranio-caudal movements of the liver, pancreas and kidneys in respiration,” Acta Radiol. Diagnosis, vol. 25, no. 2, pp. 129-131, 1984.
[15′] R. T. Poon et al., “Learning curve for radiofrequency ablation of liver tumors: prospective analysis of initial 100 patients in a tertiary institution,” Ann. Surg., vol. 239, no. 4, p. 441, 2004.
[16′] R. Zhang, S. Wu, W. Wu, H. Gao, and Z. Zhou, “Computer-assisted needle trajectory planning and mathematical modeling for liver tumor thermal ablation: A review,” Math. Biosci. Eng, vol. 16, pp. 4846-4872, 2019.
[17′] J. Kettenbach and G. Kronreif, “Robotic systems for percutaneous needle-guided interventions,” Minim. Invasive Ther. Allied Technol., vol. 24, no. 1, pp. 45-53, 2015.
[18′] M. M. Arnolli, N. C. Hanumara, M. Franken, D. M. Brouwer, and I. A. M. J. Broeders, “An overview of systems for CT- and MRI-guided percutaneous needle placement in the thorax and abdomen,” Int. J. Med. Robot. Comput. Assist. Surg., vol. 11, no. 4, pp. 458-475, 2015.
[19′] M. W. Spong, S. Hutchinson, and M. Vidyasagar, Robot modeling and control, vol. 3. Wiley New York, 2006.
[20′] Y. Lei et al., “Deep learning-based breast tumor detection and segmentation in 3D ultrasound image,” in Medical Imaging 2020: Ultrasonic Imaging and Tomography, 2020, vol. 11319, p. 113190Y.
[21′] Y. Lei, Z. Tian, S. Kahn, W. J. Curran, T. Liu, and X. Yang, “Automatic detection of brain metastases using 3D mask R-CNN for stereotactic radiosurgery,” in Medical Imaging 2020: Computer-Aided Diagnosis, 2020, vol. 11314, p. 113142X.
[22′] X. Yang et al., “A MR-TRUS registration method for ultrasound-guided prostate interventions,” in Medical Imaging 2015: Image-Guided Procedures, Robotic Interventions, and Modeling, 2015, vol. 9415, p. 94151Y.
[23′] X. Yang, P. Rossi, T. Ogunleye, A. Jani, W. Curran, and T. Liu, “TU-F-BRF-02: MR-US Prostate Registration Using Patient-Specific Tissue Elasticity Property Prior for MR-Targeted, TRUS-Guided HDR Brachytherapy,” Med. Phys., vol. 41, no. 6 Part 27, pp. 470-471, 2014.
[24′] Q. Zeng et al., “Label-driven MRI-US registration using weakly-supervised learning for MRI-guided prostate radiotherapy.,” Phys. Med. Biol., 2020.
[25′] X. Yang et al., “Prostate CT segmentation method based on nonrigid registration in ultrasound-guided CT-based HDR prostate brachytherapy,” Med. Phys., vol. 41, no. 11, p. 111915, 2014.
[26′] X. Yang, A. B. Jani, P. J. Rossi, H. Mao, W. J. Curran, and T. Liu, “A MRI-CT prostate registration using sparse representation technique,” in Medical Imaging 2016: Image-Guided Procedures, Robotic Interventions, and Modeling, 2016, vol. 9786, p. 978627.
[27′] R. L. Siegel, K. D. Miller, H. E. Fuchs, and A. Jemal, “Cancer Statistics, 2021.,” CA. Cancer J. Clin., vol. 71, no. 1, pp. 7-33, 2021.
[34′] B. J. J. Abdullah, C. H. Yeong, K. L. Goh, B. K. Yoong, G. F. Ho, C. C. W. Yim, and A. Kulkarni, “Robot-assisted radiofrequency ablation of primary and secondary liver tumours: early experience,” European radiology, vol. 24, pp. 79-85, 2014.
[35′] N. Hata, S. E. Song, O. Olubiyi, Y. Arimitsu, K. Fujimoto, T. Kato, K. Tuncali, S. Tani, and J. Tokuda, “Body-mounted robotic instrument guide for image-guided cryotherapy of renal cancer,” Medical physics, vol. 43, pp. 843-853, 2016.
[36′] N. Hata, R. Hashimoto, J. Tokuda, and S. Morikawa, “Needle guiding robot for MR-guided microwave thermotherapy of liver tumor using motorized remote-center-of-motion constraint,” in Proceedings of the 2005 IEEE International Conference on Robotics and Automation, 2005, pp. 1652-1656.
[37′] E. Franco, D. Brujic, M. Rea, W. M. Gedroyc, and M. Ristic, “Needle-guiding robot for laser ablation of liver tumors under MRI guidance,” IEEE/ASME Transactions on Mechatronics, vol. 21, pp. 931-944, 2015.
[38′] S. Tovar-Arriaga, R. Tita, J. C. Pedraza-Ortega, E. Gorrostieta, and W. A. Kalender, “Development of a robotic FD-CT-guided navigation system for needle placement-preliminary accuracy tests,” The International Journal of Medical Robotics and Computer Assisted Surgery, vol. 7, pp. 225-236, 2011.
[39′] E. Franco, M. Ristic, M. Rea, and W. M. Gedroyc, “Robot-assistant for MRI-guided liver ablation: A pilot study,” Medical physics, vol. 43, pp. 5347-5356, 2016.
[40′] R. Monfaredi, I. Iordachita, E. Wilson, R. Sze, K. Sharma, A. Krieger, S. Fricke, and K. Cleary, “Development of a shoulder-mounted robot for MRI-guided needle placement: phantom study,” International journal of computer assisted radiology and surgery, vol. 13, pp. 1829-1841, 2018.
[41′] G. Li, N. A. Patel, J. Hagemeister, J. Yan, D. Wu, K. Sharma, K. Cleary, and I. Iordachita, “Body-mounted robotic assistant for MRI-guided low back pain injection,” International Journal of Computer Assisted Radiology and Surgery, pp. 1-11, 2019.
[42′] B. Duan, R. Wen, C.-B. Chng, W. Wang, P. Liu, J. Qin, J. L. Peneyra, S. K.-Y. Chang, P.-A. Heng, and C.-K. Chui, “Image-guided robotic system for radiofrequency ablation of large liver tumor with single incision,” in 2015 12th International Conference on Ubiquitous Robots and Ambient Intelligence (URAI), 2015, pp. 284-289.
[43′] D. Stoianovici, K. Cleary, A. Patriciu, D. Mazilu, A. Stanimir, N. Craciunoiu, V. Watson, and L. Kavoussi, “AcuBot: a robot for radiological interventions,” IEEE Transactions on Robotics and Automation, vol. 19, pp. 927-930, 2003.
[44′] L. Maier-Hein, C. J. Walsh, A. Seitel, N. C. Hanumara, J.-A. Shepard, A. M. Franz, F. Pianka, S. A. Müller, B. Schmied, and A. H. Slocum, “Human vs. robot operator error in a needle-based navigation system for percutaneous liver interventions,” in Medical Imaging 2009: Visualization, Image-Guided Procedures, and Modeling, 2009, p. 72610Y.

What is claimed is:

1. A system comprising:
a robotic instrument comprising:
a needle to be employed in a procedure;
a multi-degree of freedom motion control and actuator assembly comprising a base and a movable platform coupled to the base via a set of two or more actuate-able linkages; and
a needle insertion mechanism coupled to a base of the multi-degree of freedom motion control and actuator assembly, the needle insertion mechanism being configured to (i) rotate two or more cams or rollers to move the needle, during a sensor-detected period of rest, along a delivery axis in a controlled step-wise manner that advance the needle into a pre-defined target and (ii) halt rotation of the two or more cams or rollers during a period of sensor-detected body movement.

2. The system of claim 1, wherein the robotic instrument comprises a controller, wherein the controller is configured to direct (i) the rotation of the two or more cams or rollers to move the needle, during the sensor-detected period of rest, along the delivery axis in a controlled step-wise manner that advance the needle toward the pre-defined target and (ii) halting of the rotation of the two or more cams or rollers during the period of sensor-detected body movement.

3. The system of claim 1 or 2, wherein the robotic instrument includes a central body that mounts a probe manipulation mechanism to direct an ultrasound probe to provide intraoperative image guidance for the needle insertion.

4. The system of any one of claims 1-3, wherein the needle insertion mechanism is mounted to the central body.

5. The system of any one of claims 1-4, wherein at least one of the two or more cams or rollers is adjustable via the needle insertion mechanism to accept different needles of different diameters.

6. The system of any one of claims 1-5, wherein the robotic instrument is made of a material that is compatible with an X-ray scanner, MRI, or CT scanner.

7. The system of any one of claims 1-6, wherein the robotic instrument is sized to be attached to a subject, including at least one of an abdomen region, a back region, a torso region, a head region, a pelvic region, an arm region, and a leg region.

8. The system of any one of claims 1-7, wherein the needle insertion mechanism comprises a motor (e.g., stepper, DC, AC motor) configured with PID controls.

9. The system of any one of claims 1-8, further comprising:
a navigation system configured to connect to a robot control module over a high-speed communication channel via a data communication protocol, wherein the robot control module is configured with drivers to actuate one or more motors of the needle insertion mechanism.

10. The system of claim 9, wherein the navigation system includes a deep-learning-based model (e.g., regional convolutional neural network (RCNN)) that integrates an attention-aware long short-term memory (LSTM) framework trained with low contrast and SNR ultrasound images.

11. A method comprising:
positioning, by a processor, a needle insertion mechanism via multi-degree of freedom motion control to orient a needle for insertion into a subject;
tracking, by the processor, tissue or tumor in an organ of the subject using a tracking operation based on acquired ultrasound images acquired at an insertion area of the needle; and
delivering, by the processor, the needle into the subject in a stepwise manner by rotating two or more cams or rollers of the needle insertion mechanism to move the needle along a delivery axis in stepwise increments, wherein each incremental delivery is based on the tracking.

12. The method of claim 11, further comprising:
directing (i) the rotation of the two or more cams or rollers to move the needle, during sensor-detected period of rest, along the delivery axis in a controlled step-wise manner that advances the needle toward the pre-defined target and (ii) halting of the rotation of the two or more cams or rollers during the period of sensor-detected body movement

13. The method of claim 11 or 12, wherein the robotic instrument includes a central body that mounts a probe manipulation mechanism to direct an ultrasound probe to provide intraoperative image guidance for the needle insertion.

14. The method of any one of claims 11-13, wherein the needle insertion mechanism is mounted to the central body.

15. The method of any one of claims 11-14, wherein at least one of the two or more cams or rollers is adjustable to accept different needles of different diameters.

16. The method of any one of claims 11-15, wherein the robotic instrument is made of a material that is compatible with an X-ray scanner, MRI, or CT scanner.

17. The method of any one of claims 13-16, wherein the robotic instrument is sized to be attached to a subject, including at least one of an abdomen region, a back region, a torso region, a head region, a pelvic region, an arm region, and a leg region.

18. The method of any one of claims 11-17, wherein the needle insertion mechanism comprises a stepper motor configured with PID controls.

19. The method of any one of claims 11-18 further comprising:
receiving target coordinates from a user interface for the insertion of the needle into the subject;
executing a control loop that (i) senses a period of rest or motion of the subject, (ii) directs rotation of two or more cams or rollers of the needle insertion mechanism to move the needle, during the sensor-detected period of rest, in the controlled step-wise manner that advance the needle toward the target coordinates and (ii) directs halting of the rotation of the two or more cams or rollers during a period of sensor-detected body movement;
tracking an endpoint or a landmark of the needle, or an associated assembly; and
exiting the control loop upon the endpoint or the landmark reaching the target coordinates.

20. A non-transitory computer readable medium having instructions stored thereon, wherein execution of the instructions by a processor causes the processor to control the system of any one of claims 1-10 or perform the method of any one of claims 11-19.

21. A method comprising operations for the system of any one of claims 1-10.
